(19) United States
(12) Patent Application Publication
(10) Pub. No.: US 2024/0287185 A1
(43) Pub. Date: Aug. 29, 2024

(54) FORMULATIONS OF ANTI-PD1 ANTIBODIES

(71) Applicant: Formycon AG, Planegg (DE)

(72) Inventors: Rainer Sigl, Landsberg Am Lech (DE); Katharina Maria Schott, Planegg (DE)

(73) Assignee: Formycon AG, Planegg (DE)

(21) Appl. No.: 18/573,798

(22) PCT Filed: Jun. 22, 2022
(86) PCT No.: PCT/EP22/67030

§ 371 Date: Dec. 22, 2023

(30) Foreign Application Priority Data
Jun. 23, 2021 (EP) 21181269.8
Oct. 5, 2021 (EP) 21200882.5
Apr. 29, 2022 (EP) 22170678.1

(51) Int. Cl. C07K 16/28; A61K 9/00; A61K 39/395; A61K 47/12; A61K 47/18; A61K 47/22; A61K 47/26

(52) U.S. Cl. CPC C07K 16/2818; A61K 9/0019; A61K 39/39591; A61K 47/12; A61K 47/183; A61K 47/22; A61K 47/26; C07K 2317/24; C07K 2317/76

(57) ABSTRACT

The present invention relates to pharmaceutical compositions of an anti-PD1 antibody comprising acitrate buffer, one or more amino acids other than histidine and a surfactant.

### FIELD OF THE INVENTION

The present invention relates to pharmaceutical compositions of an anti-PD1 antibody comprising a citrate buffer, one or more amino acids other than histidine and a surfactant.

### BACKGROUND OF THE INVENTION

Programmed cell death protein 1 (PD-1) is a cell surface protein which acts as an immune checkpoint and prevents autoimmune diseases by suppressing T cell inflammatory activity. PD-1 binds two ligands, PD-L1 and PD-L2. PD-L1 overexpression has been found in several cancer types and it was found that the inhibition of the interaction between PD-1 and PD-L1 can enhance T cell responses and thereby mediate antitumor activity.

Several antibodies targeting either PD-1 or PD-L1 have been developed. Pembrolizumab, marketed under the trade name Keytruda®, is a humanized IgG4 antibody which binds to PD-1 and blocks its interaction with PD-L1. It is presently authorized for the treatment of several cancer types including melanoma, non-small cell lung cancer, head and neck squamous cell carcinoma, bladder cancer and Hodgkin's lymphoma. Nivolumab, marketed under the trade name Opdivo®, is a fully human IgG4 antibody which binds to PD-1 and blocks its interaction with PD-L1. It is presently authorized for the treatment of melanoma, non-small cell lung cancer and renal cell carcinoma.

WO 2012/135408 and WO 2019/160751 A2 disclose liquid and lyophilized formulations of pembrolizumab comprising a histidine buffer, polysorbate 80 and sucrose.

WO 2017/054646 A1 discloses formulations of an anti-PD1 antibody containing sodium acetate, α,α-trehalose dihydrate and polysorbate 20, pH 5.2.

WO 2018/028383 A1 describes a pharmaceutical formulation comprising an anti-PD1 antibody, citrate, histidine, mannitol, sodium chloride, edetate and polysorbate 20 or polysorbate 80, pH 5.5 to 6.5.

WO 2018/187057 A1 discloses pharmaceutical compositions comprising an anti-PD1 antibody, histidine, sucrose, proline and polysorbate 80, pH 6.0.

WO 2018/204368 A1 describes formulations of an anti-PD1 antibody comprising a buffer, a stabilizer, a non-ionic surfactant and an anti-oxidant.

WO 2019/142149 A2 discloses pharmaceutical compositions comprising an anti-PD1 antibody, histidine, sucrose, polysorbate 20 and EDTA, pH 6.5.

WO 2019/171253 A1 describes a pharmaceutical composition comprising anti-PD1 antibody, a disaccharide, a buffer, a chelating agent and a polysorbate, pH 4.5 to 5.5.

WO 2020/097141 A1 describes a pharmaceutical composition comprising anti-PD1 antibody, a buffer, a stabilizer, a surfactant and an anti-oxidant.

WO 2021/118321 A1 discloses a pharmaceutical preparation of an anti-PD1 antibody which is free of buffer.

WO 2021/123202 describes a liquid pharmaceutical composition comprising anti-PD1 antibody, a histidine or a citrate buffer, a sugar or sugar alcohol and a non-ionic surfactant.

Nevertheless, there is still a need for stable formulations of anti-PD1 antibodies which are safe for administration to human patients.

### SUMMARY OF THE INVENTION

The present invention also relates to a liquid pharmaceutical composition comprising:
(a) an anti-human PD1 antibody;
(b) one or more amino acids other than histidine;
(c) a non-ionic surfactant;
(d) citrate buffer,
   wherein the pH of the composition is between 5.0 and 5.8, preferably is 5.5.

In one embodiment the citrate buffer is present in a concentration of 1 mM to 50 mM, preferably of 5 mM or 10 mM.

In one embodiment the non-ionic surfactant is polysorbate 20 or polysorbate 80, preferably is polysorbate 80.

In one embodiment the non-ionic surfactant is present in a concentration of 0.1 mg/ml to 0.4 mg/ml, preferably of 0.2 mg/ml.

In one embodiment the amino acid is arginine and/or lysine.

In one embodiment the pharmaceutical composition comprises arginine in a concentration of 100 mM to 350 mM or 250 mM to 350 mM.

In one embodiment the pharmaceutical composition comprises lysine in a concentration of 100 mM to 300 mM.

In one embodiment the pharmaceutical composition comprises arginine and lysine, wherein the total concentration of arginine and lysine is 100 mM to 350 mM.

In one embodiment the anti-human PD1 antibody is pembrolizumab.

In one embodiment the anti-human PD1 antibody is present in a concentration of 25 to 80 mg/ml.

The present invention also relates to a liquid pharmaceutical composition consisting of citrate buffer, arginine and/or lysine, polysorbate 20 or polysorbate 80, pembrolizumab and water for injection and having a pH of 5.0 to 5.8.

In one embodiment the liquid pharmaceutical composition consists of 5 or 10 mM citrate buffer, 300 mM arginine, 0.2 mg/ml polysorbate 80, 25 mg/ml pembrolizumab and water for injection and having a pH of 5.0 to 5.8.

In one embodiment the liquid pharmaceutical composition consists of 10 mM citrate buffer, 250 mM lysine, 0.2 mg/ml polysorbate 80, 25 mg/ml pembrolizumab and water for injection and having a pH of 5.0 to 5.8.

In one embodiment the liquid pharmaceutical composition consists of 10 mM citrate buffer, 150 mM arginine, 150 mM lysine, 0.2 mg/ml polysorbate 80, 25 mg/ml pembrolizumab and water for injection and having a pH of 5.0 to 5.8.

The present invention also relates to a liquid pharmaceutical composition consisting of 5 mM citrate buffer, 205 mM trehalose dihydrate, 0.2 mg/ml polysorbate 80, 25 mg/ml pembrolizumab and water for injection and having a pH of 5.0 to 5.8, preferably a pH of 5.5.

The present invention also relates to a liquid pharmaceutical composition consisting of L-histidine/histidine hydrochloride, sucrose or trehalose dihydrate, polysorbate 20, 90 to 350 mg/ml pembrolizumab and water for injection and having a pH of 5.2 to 5.8.

In one embodiment the liquid pharmaceutical composition consists of 10 mM L-histidine/histidine hydrochloride, 140 to 220 mM trehalose dihydrate, 0.1 mg/ml polysorbate 20, 100 or 150 mg/ml pembrolizumab and water for injection and having a pH of 5.2 to 5.8.

The present invention also relates to a liquid pharmaceutical composition consisting of citrate buffer, sucrose or trehalose dihydrate, polysorbate 80, 90 to 350 mg/ml pembrolizumab and water for injection and having a pH of 5.2 to 5.8.

The present invention also relates to a liquid pharmaceutical composition consisting of 10 mM citrate buffer, 140 to 220 mM trehalose dihydrate, 0.2 mg/ml polysorbate 80, 100 or 150 mg/ml pembrolizumab and water for injection and having a pH of 5.0 to 5.8.

The present invention also relates to a liquid pharmaceutical composition consisting of 10 mM citrate buffer, 205 mM trehalose dihydrate, 0.2 mg/ml polysorbate 80, 100 or 150 mg/ml pembrolizumab and water for injection and having a pH of 5.0 to 5.8.

In one embodiment the liquid pharmaceutical composition is for use in the treatment of cancer.

In one embodiment the cancer is melanoma, non-small cell lung carcinoma, classical Hodgkin lymphoma, urothelial carcinoma, head and neck squamous cell carcinoma, renal cell carcinoma, colorectal cancer, small cell lung cancer, microsatellite instability-high or mismatch repair deficient cancer, primary mediastinal large B-cell lymphoma, gastric or gastroesophageal junction adenocarcinoma, hepatocellular carcinoma, Merkel cell carcinoma, endometrial carcinoma, tumor mutational burden-high cancer, cutaneous squamous cell carcinoma, triple-negative breast cancer or cervical cancer.

### BRIEF DESCRIPTION OF THE DRAWINGS

FIG. 1 is a contour diagram created by the DOE software depicting the predicted HMWS peak delta (after 2 weeks storage at 40° C.) for SE-HPLC, depending on factors concentration of citric acid monohydrate and pH.

FIG. 2 is a contour diagram created by the DOE software depicting the predicted HMWS peak delta (after 2 weeks storage at 40° C.) for SE-HPLC, depending on factors pH and concentration of trehalose dihydrate.

FIG. 3 is a contour diagram created by the DOE software depicting the predicted monomer peak delta (after 2 weeks storage at 40° C.) for SE-HPLC, depending on factors concentration of citric acid monohydrate and pH.

FIG. 4 is a contour diagram created by the DOE software depicting the predicted LMWS peak delta (after 2 weeks storage at 40° C.) for SE-HPLC, depending on factors pH and concentration of trehalose dihydrate.

FIG. 5 is a contour diagram created by the DOE software depicting the predicted LMWS peak delta (after 2 weeks storage at 40° C.) for SE-HPLC, depending on factors concentration of citric acid monohydrate and pH.

FIG. 6 is a contour diagram created by the DOE software depicting the predicted LMWS peak delta (after 2 weeks storage at 40° C.) for SE-HPLC, depending on factors concentration of trehalose dihydrate and concentration of polysorbate 20 (PS20).

FIG. 7 is a contour diagram created by the DOE software depicting the predicted acidic peak delta (after 2 weeks storage at 40° C.) for CEX-HPLC, depending on factors concentration of citric acid monohydrate and pH.

FIG. 8 is a contour diagram created by the DOE software depicting the predicted acidic peak delta (after 2 weeks storage at 40° C.) for CEX-HPLC, depending on factors concentration of citric acid monohydrate and concentration of trehalose dihydrate.

FIG. 9 is a contour diagram created by the DOE software depicting the predicted main peak delta (after 2 weeks storage at 40° C.) for CEX-HPLC, depending on factors concentration of citric acid monohydrate and pH.

FIG. 10 is a contour diagram created by the DOE software depicting the predicted main peak delta (after 2 weeks storage at 40° C.) for CEX-HPLC, depending on factors pH and concentration of trehalose dihydrate.

FIG. 11 is a contour diagram created by the DOE software depicting the predicted HMWS peak delta (after 2 weeks storage at 40° C.) for SE-HPLC, depending on factors concentration of lysine HCl and concentration of arginine HCl.

FIG. 12 is a contour diagram created by the DOE software depicting the predicted monomer peak delta (after 2 weeks storage at 40° C.) for SE-HPLC, depending on factors concentration of lysine HCl and concentration of arginine HCl.

FIG. 13 is a contour diagram created by the DOE software depicting the predicted acidic peak delta (after 2 weeks storage at 40° C.) for CEX-HPLC, depending on factors concentration of lysine HCl and concentration of arginine HCl.

FIG. 14 is a contour diagram created by the DOE software depicting the predicted acidic peak delta (after 2 weeks storage at 40° C.) for CEX-HPLC, depending on factors concentration of arginine HCl and concentration of glycerol.

FIG. 15 is a contour diagram created by the DOE software depicting the predicted main peak delta (after 2 weeks storage at 40° C.) for CEX-HPLC, depending on factors concentration of lysine HCl and concentration of glycerol.

FIG. 16 is a contour diagram created by the DOE software depicting the predicted main peak delta (after 2 weeks storage at 40° C.) for CEX-HPLC, depending on factors concentration of arginine HCl and concentration of glycerol.

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments, but the invention is not limited thereto, but only by the claims.

Where the term “comprising” is used in the present description and claims, it does not exclude other elements. For the purposes of the present invention, the term “consisting of” is considered to be a preferred embodiment of the term “comprising” and excludes the presence of other elements. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a cell or organism is defined to be obtainable by a specific method, this is also to be understood to disclose a cell or organism which is obtained by this method.

Where an indefinite or definite article is used when referring to a singular noun, e.g. “a”, “an” or “the”, this includes a plural of that noun unless something else is specifically stated.

The term “pharmaceutical composition” as used herein refers to any composition comprising a chemical or biological substance or active ingredient which composition is intended for use in the medical cure, treatment, or prevention of disease and which is in such a form as to permit the active ingredient to be effective. In particular, a pharmaceutical composition does not contain excipients which are unacceptably toxic to a subject to which the composition is to be administered. The pharmaceutical compositions are sterile, i.e. aseptic and free from all living microorganisms and their spores. The pharmaceutical composition used in the present invention is liquid and stable.

In a “liquid composition” the pharmaceutically active agent, e.g. the anti-PD1 antibody, can be combined with a variety of excipients to ensure a stable active medication following storage. In one embodiment, the liquid pharmaceutical composition used in the invention is at no point lyophilized, i.e. the production method does not contain a lyophilization step and the composition is not lyophilized for storage. Liquid compositions can be stored in vials, IV bags, ampoules, cartridges, and prefilled or ready-to-use syringes.

In another embodiment the liquid composition is lyophilized after its preparation. The terms “lyophilization”, “lyophilized” and “freeze-dried” refer to a process in which the material to be dried is first frozen and then the ice or frozen solvent is removed by sublimation in a vacuum environment. Preferably, the lyophilized formulation is prepared by lyophilizing the liquid pharmaceutical composition of the present invention. The skilled person is aware of protocols for lyophilization.

A “stable” liquid composition is one in which the anti-PD1 antibody contained therein essentially retains its physical stability and/or chemical stability and/or biological activity upon storage for a certain period. Preferably, the composition essentially retains upon storage its physical and chemical stability, as well as its biological activity. Various analytical techniques for measuring protein stability are available in the art and are reviewed, for example, in Peptide and Protein Drug Delivery, 247-301, Vincent Lee Ed, Marcel Dekker, Inc, New York, New York, Pubs (1991) and Jones, Adv Drug Delivery Rev, 1993, 10:29-90. For example, stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection), by assessing charge heterogeneity using cation exchange chromatography or capillary zone electrophoresis, amino-terminal or carboxy-terminal sequence analysis, mass spectrometric analysis, SDS-PAGE analysis to detect aggregated or fragmented molecules, peptide map (for example tryptic or LYS-C) analysis, evaluating biological activity or binding of the antagonist, etc.

Preferably, the pharmaceutical composition is stable at a temperature of about 40° C. for at least 1 to 2 weeks, and/or is stable at a temperature of about 5° C. for at least 3 months, preferably 6 months or 9 months and more preferably one year, and/or is stable at a temperature of about 25° C. for at least two weeks or one month. Furthermore, the formulation is preferably stable following freezing (to, e.g., −80° C.) and thawing of the formulation at 25° C. as described in the examples herein, for example following 1, 2, 3 or 4 cycles of freezing and thawing.

For example, in the pharmaceutical composition of the present invention the percentage of high molecular weight species of the anti-PD1 antibody relative to the total amount of the anti-PD1 antibody as measured by size exclusion chromatography is not more than 10%, preferably not more than 5%, more preferably not more than 3% and most preferably not more than 2% after storage for two weeks at a temperature of about 40° C.

Alternatively, in the pharmaceutical composition of the present invention the percentage of high molecular weight species of the anti-PD1 antibody relative to the total amount of the anti-PD1 antibody as measured by size exclusion chromatography is not more than 10%, preferably not more than 5%, more preferably not more than 3% and most preferably not more than 2% after storage for one month at a temperature of about 5° C.

Alternatively, in the pharmaceutical composition of the present invention the percentage of high molecular weight species of the anti-PD1 antibody relative to the total amount of the anti-PD1 antibody as measured by size exclusion chromatography is not more than 10%, preferably not more than 5%, more preferably not more than 3% and most preferably not more than 2% after storage for three months, six months or nine months at a temperature of about 5° C.

Further, in the pharmaceutical composition of the present invention the percentage of glycated species of the anti-PD1 antibody relative to the total amount of the anti-PD1 antibody as measured by liquid chromatography-electrospray ionization-mass spectrometry (LC-ESI-MS) is not more than 2.3%, preferably not more than 2.0%, more preferably not more than 1.8% after storage for 1 month at a temperature of about 40° C.

A “buffer” is an aqueous solution consisting of a mixture of a weak acid and its conjugate base or vice versa which resists changes in its pH and therefore keeps the pH at a nearly constant value. The buffer of the present invention preferably has a pH in the range from about 5.0 to about 5.8, preferably from about 5.2 to about 5.7, more preferably from about 5.4 to 5.7 and most preferably has a pH of about 5.5.

The buffer used in the present invention is a histidine-containing buffer or a citrate buffer. In one embodiment, the citrate buffer is the only buffer present in the pharmaceutical composition of the present invention. Preferably, the histidine-containing buffer is the only buffer present in the liquid pharmaceutical composition of the present invention. In particular, the liquid pharmaceutical composition of the present invention does not comprise a mixture of a histidine buffer and a citrate buffer.

In one embodiment, the buffer is a citrate buffer. The citrate buffer is prepared by mixing citric acid with a citrate salt such as sodium citrate or by mixing citric acid with a base such as sodium hydroxide, arginine and/or lysine. The citrate buffer has a concentration of 1 mM to 50 mM, preferably of 2 mM to 40 mM, more preferably of 3 mM to 30 mM, even more preferably of 4 mM to 20 mM and most preferably of 5 mM to 10 mM. In one embodiment, the citrate buffer has a concentration of 5 mM. In one embodiment, the citrate buffer has a concentration of 10 mM. In one embodiment, the citrate buffer has a concentration of 2 mM.

According to the invention the citrate buffer has a pH in the range from about 5.0 to 5.8, preferably from about 5.1 to 5.7, more preferably of about 5.2 to 5.6 or about 5.3 to 5.6 and most preferably has a pH of about 5.5.

In one embodiment, the citrate buffer comprises citric acid and sodium citrate in a concentration of 10 mM. In another embodiment the citrate buffer comprises citric acid and sodium citrate in a concentration of 10 mM and with a pH of 5.5.

In one embodiment, the citrate buffer has a concentration of 10 mM and a pH of 5.5.

In one embodiment, the citrate buffer comprises citric acid and citrate in a concentration of 10 mM. In another embodiment the citrate buffer comprises citric acid and citrate in a concentration of 10 mM and with a pH of 5.5. The pH may be adjusted to pH 5.5 by adding HCl/sodium hydroxide, arginine/arginine HCl and/or lysine/lysine HCl.

In one embodiment, the citrate buffer comprises citric acid and sodium citrate in a concentration of 5 mM. In another embodiment the citrate buffer comprises citric acid and sodium citrate in a concentration of 5 mM and with a pH of 5.5.

In one embodiment, the citrate buffer comprises citric acid and citrate in a concentration of 5 mM. In another embodiment the citrate buffer comprises citric acid and citrate in a concentration of 5 mM and with a pH of 5.5. The pH may be adjusted to pH 5.5 by adding HCl/sodium hydroxide, arginine/arginine HCl and/or lysine/lysine HCl.

The terms “histidine-containing buffer” and “histidine buffer” are used interchangeably herein and refer to a buffer comprising histidine. Examples of histidine buffers include histidine chloride, histidine hydrochloride, histidine acetate, histidine phosphate, and histidine sulphate. The preferred histidine buffer of the invention further comprises L-histidine. Even more preferably the histidine buffer of the invention comprises histidine hydrochloride, most preferably it comprises histidine hydrochloride and L-histidine.

Preferably, the histidine buffer or histidine hydrochloride buffer or histidine hydrochloride/L-histidine buffer has a pH in the range from about 5.2 to 5.8, preferably from about 5.3 to 5.7, more preferably of 5.4 to 5.6 and most preferably has a pH of about 5.5.

In a particular preferred embodiment, the histidine-containing buffer comprises histidine hydrochloride/L-histidine in a concentration of 5 to 30 mM, preferably of 7 to 20 mM, more preferably of 8 to 15 mM and most preferably of 10 mM.

In another particular preferred embodiment the buffer is histidine hydrochloride/L-histidine with a concentration of 10 mM and with a pH of 5.5.

A “surfactant” as used herein refers to an amphiphilic compound, i.e. a compound containing both hydrophobic groups and hydrophilic groups which lowers the surface tension (or interfacial tension) between two liquids or between a liquid and a solid. A “non-ionic surfactant” has no charged groups in its head. The formation of insoluble particles during freeze/thaw cycles of antibody-containing compositions can be remarkably inhibited by addition of surfactants. Examples of “non-ionic surfactants” include e.g. polyoxyethylene glycol alkyl ethers, such as octaethylene glycol monododecyl ether, pentaethylene glycol monododecyl ether; polyoxypropylene glycol alkyl ethers; glucoside alkyl ethers, such as decyl glucoside, lauryl glucoside, octyl glucoside; polyoxyethylene glycol octylphenol ethers, such as triton X-100; polyoxyethylene glycol alkylphenol ethers, such as nonoxynol-9; glycerol alkyl esters, such as glyceryl laurate; polyoxyethylene glycol sorbitan alkyl esters, such as polysorbate; sorbitan alkyl esters, such as spans; cocamide MEA, cocamide DEA, dodecyldimethylamine oxide; block copolymers of polyethylene glycol and polypropylene glycol, such as poloxamers; and polyethoxylated tallow amine (POEA). The liquid pharmaceutical compositions of the present invention can contain one or more of these surfactants in combination. In a preferred embodiment the pharmaceutical compositions of the present invention contain only one non-ionic surfactant.

Preferred non-ionic surfactants for use in the pharmaceutical compositions of the present invention are polysorbates such as polysorbate 20, 40, 60 or 80, and especially polysorbate 20 (i.e. Tween 20) or polysorbate 80 (i.e. Tween 80).

The concentration of the non-ionic surfactant is in the range of 0.005 to 0.06% (w/v), preferably in the range of 0.008 to 0.05% (w/v), and most preferably in the range of 0.01 to 0.04% (w/v), relative to the total volume of the composition.

In a preferred embodiment, the non-ionic surfactant is polysorbate 20. In a preferred embodiment, the non-ionic surfactant is polysorbate 20 with a concentration in the range of 0.05 to 0.6 mg/ml, preferably in the range of 0.08 to 0.5 mg/ml, and most preferably in the range of 0.1 to 0.4 mg/ml. In a preferred embodiment, the non-ionic surfactant is polysorbate 20 with a concentration of 0.1 mg/ml. In a preferred embodiment, the non-ionic surfactant is polysorbate 20 with a concentration of 0.2 mg/ml.

In another preferred embodiment, the non-ionic surfactant is polysorbate 80 with a concentration in the range of 0.05 to 0.6 mg/ml, preferably in the range of 0.08 to 0.5 mg/ml, more preferably in the range of 0.1 to 0.4 mg/ml and most preferably of 0.2 mg/ml.

In a particularly preferred embodiment, the non-ionic surfactant is polysorbate 80 with a concentration of 0.2 mg/ml.

In one embodiment, the non-ionic surfactant is polysorbate 20 with a concentration of 0.4 mg/ml. In one embodiment, the non-ionic surfactant is polysorbate 20 with a concentration of 0.1 mg/ml.

In one embodiment, the pharmaceutical composition comprising an anti-human PD1 antibody, citrate buffer, a non-ionic surfactant and one or more amino acids other than histidine does not comprise a sugar. In one embodiment, the pharmaceutical composition comprising an anti-human PD1 antibody, citrate buffer, a non-ionic surfactant and one or more amino acids other than histidine does not comprise sucrose. In one embodiment, the pharmaceutical composition comprising an anti-human PD1 antibody, citrate buffer, a non-ionic surfactant and one or more amino acids other than histidine does not comprise trehalose.

The liquid pharmaceutical composition of the present invention comprises one or more amino acids other than histidine. Amino acids are organic compounds that contain amino and carboxyl groups and a side chain which is specific for each amino acid. Amino acids which can be present in the liquid pharmaceutical composition of the present invention may be selected from the group consisting of arginine, lysine, aspartic acid, glutamic acid, serine, threonine, asparagine, glutamine, cysteine, glycine, proline, alanine, valine, isoleucine, methionine, phenylalanine, tyrosine and tryptophan. In one embodiment, the liquid pharmaceutical composition of the present invention comprises arginine. In one embodiment, the liquid pharmaceutical composition of the present invention comprises lysine. In one embodiment, the liquid pharmaceutical composition of the present invention comprises arginine and lysine. In one embodiment, arginine is the only amino acid present in the liquid pharmaceutical composition of the present invention. In one embodiment, lysine is the only amino acid present in the liquid pharmaceutical composition of the present invention. In one embodiment, arginine and lysine are the only amino acids present in the liquid pharmaceutical composition of the present invention.

The arginine and/or lysine may be added to the liquid pharmaceutical composition as the free base or as the hydrochloride salt thereof. In one embodiment, the arginine is added as a mixture of the free base arginine with the hydrochloride salt of arginine, i.e. arginine hydrochloride (arginine-HCl). In one embodiment, the lysine is added as a mixture of the free base lysine with the hydrochloride salt of lysine, i.e. lysine hydrochloride (lysine-HCl). In one embodiment, the arginine is added as a mixture of the free base arginine with the hydrochloride salt of arginine, i.e. arginine hydrochloride, and the lysine is added as a mixture of the free base lysine with the hydrochloride salt of lysine, i.e. lysine hydrochloride.

In one embodiment, arginine is added as a mixture of the free base arginine with the hydrochloride salt of arginine, i.e. arginine hydrochloride (arginine-HCl) and the ratio of free base arginine to arginine-HCl is 1:5 to 1:12, preferably the ratio is 1:7 to 1:12, more preferably the ratio is 1:8 to 1:12 even more preferably the ratio is 1:9 to 1:11 and most preferably the ratio is 1:10.5.

In one embodiment, the liquid pharmaceutical composition of the present invention comprises arginine in a concentration of 100 mM to 350 mM, preferably of 120 mM to 330 mM, more preferably of 130 mM to 320 mM and most preferably of 150 mM to 300 mM. In one embodiment, the liquid pharmaceutical composition of the present invention comprises arginine in a concentration of 250 mM to 350 mM, preferably of 260 mM to 340 mM, more preferably of 270 mM 25 to 330 mM, even more preferably of 280 mM to 320 mM and most preferably of 290 mM to 310 mM. In one embodiment, the liquid pharmaceutical composition of the present invention comprises arginine in a concentration of 150 mM. In one embodiment, the liquid pharmaceutical composition of the present invention comprises arginine in a concentration of 250 mM. In one embodiment, the liquid pharmaceutical composition of the present invention comprises arginine in a concentration of 300 mM. In one embodiment, the liquid pharmaceutical composition of the present invention comprises 26 mM L-arginine and 274 mM L-arginine-HCl.

In one embodiment, the liquid pharmaceutical composition of the present invention comprises lysine in a concentration of 100 mM to 300 mM, preferably of 120 mM to 280 mM, more preferably of 130 mM to 270 mM and most preferably of 150 mM to 250 mM. In one embodiment, the liquid pharmaceutical composition of the present invention comprises lysine in a concentration of 150 mM. In one embodiment, the liquid pharmaceutical composition of the present invention comprises lysine in a concentration of 250 mM.

In one embodiment, the liquid pharmaceutical composition of the present invention comprises arginine and lysine in a total concentration of 100 mM to 350 mM, preferably of 150 mM to 330 mM, more preferably of 180 mM to 310 mM and most preferably of 300 mM.

In one embodiment, the liquid pharmaceutical composition of the present invention comprises 100 mM to 200 mM arginine and 100 mM to 200 mM lysine. In one embodiment, the liquid pharmaceutical composition of the present invention comprises 120 mM to 180 mM arginine and 120 mM to 180 mM lysine. In one embodiment, the liquid pharmaceutical composition of the present invention comprises 130 mM to 170 mM arginine and 130 mM to 170 mM lysine. In one embodiment, the liquid pharmaceutical composition of the present invention comprises 140 mM to 160 mM arginine and 140 mM to 160 mM lysine. In one embodiment, the liquid pharmaceutical composition of the present invention comprises 150 mM arginine and 150 mM lysine.

In one embodiment the liquid pharmaceutical composition of the present invention comprises a sugar alcohol. Sugar alcohols are organic compounds derived from a sugar which contain a hydroxyl group attached to each carbon atom. Suitable sugar alcohols include glycerol, mannitol, sorbitol, and xylitol.

Preferably, the sugar alcohol is glycerol. The concentration of glycerol in the liquid pharmaceutical composition of the present invention is 50 mM to 200 mM, preferably the concentration of glycerol is 60 mM to 180 mM, more preferably the concentration of mannitol or sorbitol is 70 mM to 150 mM or 80 mM to 120 mM and most preferably the concentration of glycerol is 100 mM. In one embodiment the liquid pharmaceutical composition of the present invention comprises 100 mM glycerol.

In one embodiment the liquid pharmaceutical composition of the present invention does not comprise mannitol. In one embodiment the liquid pharmaceutical composition of the present invention does not comprise sorbitol.

In one embodiment, the liquid pharmaceutical composition of the present invention does not contain sodium chloride. In one embodiment, the liquid pharmaceutical composition of the present invention does not contain any sodium salt. In one embodiment, the liquid pharmaceutical composition of the present invention does not contain any inorganic salt. As used herein, an “inorganic salt” refers to an ionic compound which has osmoregulatory properties. An inorganic salt such as sodium chloride (NaCl) can dissociate in solution into its constituent ions, i.e. NaCl dissociates into Na+ and Cl− ions, which both affect the osmotic pressure, i.e. the osmolality, of the solution. Exemplary inorganic salts which are not present in the liquid pharmaceutical composition of the present invention are potassium chloride, calcium chloride, sodium chloride, sodium phosphate, potassium phosphate and sodium bicarbonate.

In one embodiment, the liquid pharmaceutical composition of the present invention does not contain EDTA. In one embodiment, the liquid pharmaceutical composition of the present invention does not contain pentetic acid. In one embodiment, the liquid pharmaceutical composition of the present invention does not contain EDTA and pentetic acid. In one embodiment, the liquid pharmaceutical composition of the present invention does not contain any chelating agent. Chelating agents can form at least one bond with a metal atom. A chelating agent is typically a multidentate ligand that can be used in compositions as a stabilizer to complex with species, which might otherwise promote instability. Exemplary chelating agents include aminopolycarboxylic acids, hydroxyaminocarboxylic acids, N-substituted glycines, 2-(2-amino-2-oxoethyl) aminoethane sulfonic acid (BES), deferoxamine (DEF), niacinamide, desoxycholates, ethylenediaminetetraacetic acid (EDTA), diethylenetriaminepentaacetic acid (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), N-hydroxyethyliminodiacetic acid (HIMDA), N,N-bis-hydroxyethylglycine (bicine), N-(trishydroxymethylmethyl) glycine (tricine), glycylglycine, sodium desoxycholate, ethylenediamine; propylenediamine; diethylenetriamine; triethylenetetraamine (trien), ethylenediaminetetraaceto EDTA; disodium EDTA, EDTA, calcium EDTA oxalic acid and malate. In the present invention histidine and citrate are not considered as chelating agents.

In one embodiment, the liquid pharmaceutical composition of the present invention does not contain methionine. In one embodiment, the liquid pharmaceutical composition of the present invention does not contain L-methionine or L-methionine-HCl. In one embodiment, the liquid pharmaceutical composition of the present invention does not contain any anti-oxidant. Anti-oxidants are compounds that inhibit oxidation by reacting with oxidizing agents. In the present invention histidine is not considered as anti-oxidant.

In one embodiment, the liquid pharmaceutical composition of the present invention does not contain proline. In one embodiment, the liquid pharmaceutical composition of the present invention does not contain glycine. In one embodiment, the liquid pharmaceutical composition of the present invention does not contain glutamic acid. In one embodiment, the liquid pharmaceutical composition of the present invention does not contain serine. In one embodiment, the liquid pharmaceutical composition of the present invention does not contain tyrosine. In one embodiment, the liquid pharmaceutical composition of the present invention does not contain tryptophan. In one embodiment, the liquid pharmaceutical composition of the present invention does not contain leucine. In one embodiment, the liquid pharmaceutical composition of the present invention does not contain phenylalanine. In one embodiment, the liquid pharmaceutical composition of the present invention does not contain threonine. In one embodiment, the liquid pharmaceutical composition of the present invention does not contain aspartate. In one embodiment, the liquid pharmaceutical composition of the present invention does not contain asparagine. In one embodiment, the liquid pharmaceutical composition of the present invention does not contain glutamine. In one embodiment, the liquid pharmaceutical composition of the present invention does not contain alanine. In one embodiment, the liquid pharmaceutical composition of the present invention does not contain cysteine. In one embodiment, the liquid pharmaceutical composition of the present invention does not contain isoleucine. In one embodiment, the liquid pharmaceutical composition of the present invention does not contain valine. In one embodiment, the liquid pharmaceutical composition of the present invention does not contain any amino acid in addition to arginine. In one embodiment, the liquid pharmaceutical composition of the present invention does not contain any amino acid in addition to lysine. In one embodiment, the liquid pharmaceutical composition of the present invention does not contain any amino acid in addition to arginine and lysine.

In one embodiment, the liquid pharmaceutical composition of the present invention does not contain EDTA and proline. In one embodiment, the liquid pharmaceutical composition of the present invention does not contain DTPA and methionine.

The term “sugar” refers to an organic compound comprising only carbon, hydrogen, and oxygen, usually with a hydrogen:oxygen atom ratio of 2:1 and the empirical formula Cm(H2O)n. The term “sugar” includes mono-, di-, oligo- and polysaccharides. Examples of sugars include glucose, fructose, galactose, xylose, ribose, sucrose, mannose, lactose, maltose, trehalose, starch, and glycogen. Preferably, the sugar is a non-reducing sugar. Non-reducing sugars are sugars which are not able to act as a reducing agent, as they do not comprise a free aldehyde or ketone group. Preferably, the non-reducing sugar is selected from sucrose and trehalose.

In one embodiment, the sugar is trehalose. Trehalose is a non-reducing sugar. It is a disaccharide formed by a 1,1-glycosidic bond between a glucose and a fructose unit. Preferably, the dihydrate form of trehalose is used. The concentration of trehalose dihydrate in the liquid pharmaceutical composition of the present invention is 100 mM to 300 mM, preferably the concentration of trehalose dihydrate is 120 mM to 280 mM, more preferably the concentration of trehalose dihydrate is 150 mM to 250 mM or 150 mM to 205 mM and most preferably the concentration of trehalose dihydrate is 150 mM or 205 mM.

In one embodiment, the sugar is sucrose. Sucrose is a non-reducing sugar. It is a disaccharide formed by a 1,2-glycosidic bond between two α-glucose units. The concentration of sucrose in the liquid pharmaceutical composition of the present invention is 100 mM to 300 mM, preferably the concentration of sucrose is 120 mM to 280 mM, more preferably the concentration of sucrose is 150 mM to 250 mM and most preferably the concentration of sucrose is 205 mM. A sucrose concentration of 205 mM equals to 70 mg/ml sucrose.

The term “antibody” or “immunoglobulin” is used herein in the broadest sense and includes full length antibodies, genetically engineered antibodies, recombinant antibodies, multivalent antibodies, monoclonal antibodies, polyclonal antibodies, bispecific antibodies, multispecific antibodies, chimeric antibodies, humanized antibodies, fully human antibodies, as well as fragments of such antibodies as long as they remain functional and exhibit the desired biological activity. The “biological activity” of an antibody refers to the ability of the antibody to bind to antigen and result in a biological response which can be measured in vitro or in vivo.

A full length antibody comprises an antigen-binding variable region of the light (VL) and heavy chain (VH), a light chain constant region (CL) and heavy chain constant domains CH1, CH2 and CH3.

The term “antibody fragment” or “antigen-binding fragment” is used herein in the broadest sense and comprises a portion of a full length antibody, preferably comprising the antigen-binding or variable region thereof. An antibody fragment retains the original specificity of the parent immunoglobulin. Examples of antibody fragments include, e.g., Fab, Fab′, F(ab′)2, and Fv fragments, diabodies, linear antibodies, single-chain antibody molecules, and multispecific antibodies formed from antibody fragment(s).

A “monoclonal antibody” is an antibody that is specific for a single epitope of an antigen, i.e. directed against a single determinant on an antigen. Methods for producing monoclonal antibodies are known to the person skilled in the art.

The term “recombinant antibody” refers to all antibodies prepared, expressed, created or isolated by recombinant means, such as antibodies isolated from a transgenic host cell, such as e.g. a NSO or CHO cell, or from an animal transgenic for immunoglobulin genes, or antibodies expressed using recombinant expression vectors transfected into a host cell, such as e.g. SP 2/0 mouse myeloma cells.

A “humanized antibody” is a human antibody wherein the antigen binding portion (CDR) is derived from non-human species, such as a mouse, and thus has a different specificity compared to the parent immunoglobulin. The CDR protein sequences can be modified to increase their similarities to antibody variants produced naturally in humans.

A “fully human antibody” is an antibody wherein all parts of the antibody including the antigen binding portion (CDR) are derived from human.

The term “anti-PD1 antibody” refers to an antibody that specifically binds to cell death protein 1 (PD-1) and inhibits the binding of PD-1 to its ligand PD-L1 and optionally inhibits the binding of PD-1 to its ligands PD-L1 and PD-L2. The anti-PD1 antibody thereby abolishes the suppressive effect of the PD-1/PD-L1 interaction on T cells. Known anti-PD1 antibodies include, but are not limited to, pembrolizumab, nivolumab, cemiplimab and cetrelimab.

Pembrolizumab (also known as MK-3475, SCH 900475 and lambrolizumab) is a humanized IgG4 mAb with the structure described in WHO Drug Information, Vol. 27, No. 2, pages 161-162 (2013) and which comprises the heavy and light chain amino acid sequences and CDRs described in Table 2 of WO 2018/204368. Pembrolizumab has been approved inter alia for the treatment of patients with unresectable or metastatic melanoma and for the treatment of certain patients with recurrent or metastatic head and neck squamous cell cancer (HNSCC), classical Hodgkin lymphoma (cHL), urothelial carcinoma, gastric cancer, microsatellite instability-high (MSI-H) cancer and non-small cell lung cancer. The present commercial pembrolizumab formulation contains 10 mM histidine, 70 mg/ml sucrose, 0.2 mg/ml polysorbate 80 and water for injection, pH 5.5 and is supplied in a concentration of 25 mg/ml.

Nivolumab (also known as ONO-4538, BMS-936558, MDX1106) is a fully human monoclonal IgG4 antibody which comprises the heavy and light chain amino acid sequences and CDRs described in Table 2 of WO 2018/204368. Pembrolizumab has been approved for the treatment of patients with melanoma, renal carcinoma, non-small cell lung cancer and urothelial carcinoma. The present commercial nivolumab formulation contains 30 mg/ml mannitol, 0.008 mg/ml pentetic acid, 0.2 mg/ml polysorbate 80, 2.92 mg/ml sodium chloride, 5.88 mg/ml sodium citrate dihydrate, and water for injection, pH 6.0 and is supplied in a concentration of 10 mg/ml.

In one embodiment, the liquid pharmaceutical composition does not contain an anti-LAG3 antibody. In one embodiment, the antibody is not a bispecific antibody.

In one embodiment, the anti-human PD-1 antibody is the only antibody present in the liquid pharmaceutical composition. In one embodiment, pembrolizumab is the only antibody present in the liquid pharmaceutical composition.

In one embodiment, the anti-human PD-1 antibody is the only pharmaceutically active agent present in the liquid pharmaceutical composition. In one embodiment, pembrolizumab is the only pharmaceutically active agent present in the liquid pharmaceutical composition.

The concentration of the anti-PD1 antibody in the pharmaceutical compositions of the present invention is typically 10-80 mg/ml, preferably 15-70 mg/ml or 15-60 mg/ml, more preferably 20-50 mg/ml or 20-40 mg/ml, and most preferably 25 mg/ml.

The pharmaceutical compositions of the present invention can be used in the treatment of cancer, in particular in the treatment of melanoma, non-small cell lung carcinoma, classical Hodgkin lymphoma, urothelial carcinoma, head and neck squamous cell carcinoma, renal cell carcinoma, colorectal cancer, oesophageal carcinoma, small cell lung cancer, microsatellite instability-high or mismatch repair deficient cancer, primary mediastinal large B-cell lymphoma, gastric or gastroesophageal junction adenocarcinoma, hepatocellular carcinoma, Merkel cell carcinoma, endometrial carcinoma, tumor mutational burden-high cancer, cutaneous squamous cell carcinoma, triple-negative breast cancer or cervical cancer.

The pharmaceutical compositions of the present invention may contain further active agents, in particular further anti-tumor agents such as chemotherapeutics. Examples of such chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, es tram us tine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomopholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY1 17018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

In an alternative embodiment, the pharmaceutical compositions of the present invention may be administered in combination with any of the chemotherapeutics listed above, but the chemotherapeutic is present in a separate pharmaceutical composition.

In an alternative embodiment, the pharmaceutical composition of the present invention is to be administered with pemetrexed and platinum-based chemotherapy. In an alternative embodiment, the pharmaceutical composition of the present invention is to be administered with pemetrexed and platinum-based chemotherapy in the treatment of non-small cell lung carcinoma. In an alternative embodiment, the pharmaceutical composition of the present invention is to be administered with carboplatin and either paclitaxel or nab-paclitaxel. In an alternative embodiment, the pharmaceutical composition of the present invention is to be administered with carboplatin and either paclitaxel or nab-paclitaxel in the treatment of non-small cell lung carcinoma.

In an alternative embodiment, the pharmaceutical composition of the present invention is to be administered with platinum-based chemotherapy and 5-fluorouracil. In an alternative embodiment, the pharmaceutical composition of the present invention is to be administered with platinum-based chemotherapy and 5-fluorouracil in the treatment of head and neck squamous cell carcinoma.

In an alternative embodiment, the pharmaceutical composition of the present invention is to be administered with axitinib. In an alternative embodiment, the pharmaceutical composition of the present invention is to be administered with axitinib in the treatment of renal cell carcinoma.

In an alternative embodiment, the pharmaceutical composition of the present invention is to be administered with platinum- and fluoropyrimidine-based chemotherapy. In an alternative embodiment, the pharmaceutical composition of the present invention is to be administered with platinum- and fluoropyrimidine-based chemotherapy in the treatment of esophageal carcinoma.

In an alternative embodiment, the pharmaceutical composition of the present invention is to be administered with lenvatinib. In an alternative embodiment, the pharmaceutical composition of the present invention is to be administered with lenvatinib in the treatment of endometrial carcinoma.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of an anti-human PD1 antibody; 100 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of an anti-human PD1 antibody; 250 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of an anti-human PD1 antibody; 280 mM to 320 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 300 mM lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 350 mM of arginine and lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 200 mM of arginine; 100 mM to 200 mM of lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of an anti-human PD1 antibody; 250 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of an anti-human PD1 antibody; 280 mM to 320 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 300 mM lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 350 mM of arginine and lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 200 mM of arginine; 100 mM to 200 mM of lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of an anti-human PD1 antibody; 100 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of an anti-human PD1 antibody; 250 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of an anti-human PD1 antibody; 280 mM to 320 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of an anti-human PD1 antibody; 100 mM to 300 mM lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of an anti-human PD1 antibody; 100 mM to 350 mM of arginine and lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of an anti-human PD1 antibody; 100 mM to 200 mM of arginine; 100 mM to 200 mM of lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of an anti-human PD1 antibody; 100 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of an anti-human PD1 antibody; 250 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of an anti-human PD1 antibody; 280 mM to 320 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of an anti-human PD1 antibody; 100 mM to 300 mM lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of an anti-human PD1 antibody; 100 mM to 350 mM of arginine and lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of an anti-human PD1 antibody; 100 mM to 200 mM of arginine; 100 mM to 200 mM of lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of pembrolizumab; 100 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of pembrolizumab; 250 mM to 350 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of pembrolizumab; 280 mM to 320 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of pembrolizumab; 100 mM to 300 mM lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of pembrolizumab; 100 mM to 350 mM of arginine and lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of pembrolizumab; 100 mM to 200 mM of arginine; 100 mM to 200 mM of lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 25 mg/ml of pembrolizumab; 100 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of pembrolizumab; 250 mM to 350 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of pembrolizumab; 280 mM to 320 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of pembrolizumab; 100 mM to 300 mM lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of pembrolizumab; 100 mM to 350 mM of arginine and lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of pembrolizumab; 100 mM to 200 mM of arginine; 100 mM to 200 mM of lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 25 mg/ml of pembrolizumab; 100 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of pembrolizumab; 250 mM to 350 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of pembrolizumab; 280 mM to 320 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of pembrolizumab; 100 mM to 300 mM lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of pembrolizumab; 100 mM to 350 mM of arginine and lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of pembrolizumab; 100 mM to 200 mM of arginine; 100 mM to 200 mM of lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 25 mg/ml of pembrolizumab; 100 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of pembrolizumab; 250 mM to 350 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of pembrolizumab; 280 mM to 320 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of pembrolizumab; 100 mM to 300 mM lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of pembrolizumab; 100 mM to 350 mM of arginine and lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 25 mg/ml of pembrolizumab; 100 mM to 200 mM of arginine; 100 mM to 200 mM of lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 300 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 300 mM of arginine and lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 150 mM of arginine; 150 mM of lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 300 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 300 mM of arginine and lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 150 mM of arginine; 150 mM of lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 300 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 250 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 250 mM mM lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 300 mM of arginine and lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 150 mM of arginine; 150 mM of lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 300 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 250 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 250 mM lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 300 mM of arginine and lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 150 mM of arginine; 150 mM of lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 300 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 250 mM of arginine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 250 mM lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 300 mM of arginine and lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 150 mM of arginine; 150 mM of lysine; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 300 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 250 mM of arginine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 250 mM lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 300 mM of arginine and lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 150 mM of arginine; 150 mM of lysine; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 300 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 250 mM of arginine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 250 mM lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 300 mM of arginine and lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 150 mM of arginine; 150 mM of lysine; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 300 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 250 mM of arginine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 250 mM lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 300 mM of arginine and lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 150 mM to 200 mM of arginine; 150 mM to 200 mM of lysine; 0.1 mg/ml to 0.4 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.2 mg/ml of a non-ionic surfactant and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.2 mg/ml of polysorbate 80 and 1 to 50 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 300 mM lysine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.2 mg/ml of a non-ionic surfactant and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 300 mM lysine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.2 mg/ml of polysorbate 80 and 1 to 50 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine and lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine and lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of an anti-human PD1 antibody; 250 mM to 350 mM of arginine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of an anti-human PD1 antibody; 280 mM to 320 mM of arginine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of an anti-human PD1 antibody; 100 mM to 300 mM lysine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of an anti-human PD1 antibody; 100 mM to 350 mM of arginine and lysine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of an anti-human PD1 antibody; 100 mM to 200 mM of arginine; 100 mM to 200 mM of lysine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of a non-ionic surfactant and 5 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of a non-ionic surfactant and 10 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of pembrolizumab; 100 mM to 350 mM of arginine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of pembrolizumab; 250 mM to 350 mM of arginine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of pembrolizumab; 280 mM to 320 mM of arginine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of pembrolizumab; 100 mM to 300 mM lysine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of pembrolizumab; 100 mM to 350 mM of arginine and lysine; 0.1 mg/ml to 0.4 mg/ml of polysorbate 80 and 10 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of polysorbate 80 and 5 mM of citrate buffer, pH 5.5.

In one embodiment, the present invention relates to a liquid pharmaceutical composition comprising: 10 to 80 mg/ml of pembrolizumab; 100 mM to 200 mM of arginine; 100 mM to 200 mM of lysine; 0.1 mg/ml to 0.4 mg/ml of polysorbate 80 and 10 mM of citrate buffer, pH 5.5.

In one embodiment, the present invention relates to a liquid pharmaceutical composition comprising: 25 mg/ml of an anti-PD1 antibody; 300 mM arginine; 0.2 mg/ml polysorbate 80 and 5 or 10 mM of citrate buffer, pH 5.0 to 5.8.

In one embodiment, the present invention relates to a liquid pharmaceutical composition comprising: 25 mg/ml of an anti-PD1 antibody; 300 mM arginine; 0.2 mg/ml polysorbate 80 and 5 or 10 mM of citrate buffer, pH 5.5.

In one embodiment, the present invention relates to a liquid pharmaceutical composition comprising: 25 mg/ml of pembrolizumab; 300 mM arginine; 0.2 mg/ml polysorbate 80 and 5 or 10 mM of citrate buffer, pH 5.0 to 5.8.

In one embodiment, the present invention relates to a liquid pharmaceutical composition comprising: 25 mg/ml of pembrolizumab; 300 mM arginine; 0.2 mg/ml polysorbate 80 and 5 or 10 mM of citrate buffer, pH 5.5.

In one embodiment, the present invention relates to a liquid pharmaceutical composition comprising: 25 mg/ml of an anti-PD1 antibody; 250 mM lysine; 0.2 mg/ml polysorbate 80 and 10 mM of citrate buffer, pH 5.0 to 5.8.

In one embodiment, the present invention relates to a liquid pharmaceutical composition comprising: 25 mg/ml of an anti-PD1 antibody; 250 mM lysine; 0.2 mg/ml polysorbate 80 and 10 mM of citrate buffer, pH 5.5.

In one embodiment, the present invention relates to a liquid pharmaceutical composition comprising: 25 mg/ml of pembrolizumab; 250 mM lysine; 0.2 mg/ml polysorbate 80 and 10 mM of citrate buffer, pH 5.0 to 5.8.

In one embodiment, the present invention relates to a liquid pharmaceutical composition comprising: 25 mg/ml of pembrolizumab; 250 mM lysine; 0.2 mg/ml polysorbate 80 and 10 mM of citrate buffer, pH 5.5.

In one embodiment, the present invention relates to a liquid pharmaceutical composition comprising: 25 mg/ml of an anti-PD1 antibody; 150 mM arginine; 150 mM lysine; 0.2 mg/ml polysorbate 80 and 10 mM of citrate buffer, pH 5.0 to 5.8.

In one embodiment, the present invention relates to a liquid pharmaceutical composition comprising: 25 mg/ml of an anti-PD1 antibody; 150 mM arginine; 150 mM lysine; 0.2 mg/ml polysorbate 80 and 10 mM of citrate buffer, pH 5.5.

In one embodiment, the present invention relates to a liquid pharmaceutical composition comprising: 25 mg/ml of pembrolizumab; 150 mM arginine; 150 mM lysine; 0.2 mg/ml polysorbate 80 and 10 mM of citrate buffer, pH 5.0 to 5.8.

In one embodiment, the present invention relates to a liquid pharmaceutical composition comprising: 25 mg/ml of pembrolizumab; 150 mM arginine; 150 mM lysine; 0.2 mg/ml polysorbate 80 and 10 mM of citrate buffer, pH 5.5.

In one embodiment, the present invention relates to a liquid pharmaceutical composition comprising: 25 mg/ml of pembrolizumab; 300 mM arginine/arginine HCl; 0.2 mg/ml polysorbate 80 and 10 mM of citric acid monohydrate, pH 5.5.

In one embodiment, the present invention relates to a liquid pharmaceutical composition comprising: 25 mg/ml of pembrolizumab; 26 mM L-arginine; 274 mM L-arginine HCl; 0.2 mg/ml polysorbate 80 and 10 mM of citric acid monohydrate, pH 5.5.

The pharmaceutical compositions may be supplied in a vial or in a pre-filled syringe. The pharmaceutical compositions may be administered by intravenous infusion, e.g. over a period of 30 minutes or less.

Alternatively, the pharmaceutical compositions may be administered by subcutaneous injection. In this case, the concentration of the anti-PD1 antibody and preferably of pembrolizumab is 80 mg/ml to 180 mg/ml, more preferably 90 mg/ml to 170 mg/ml and most preferably 100 mg/ml to 165 mg/ml. In one embodiment, the concentration of the anti-PD1 antibody and preferably of pembrolizumab is 100 mg/ml, 150 mg/ml or 165 mg/ml.

In one embodiment, the present invention relates to a liquid pharmaceutical composition comprising: 90 mg/ml to 350 mg/ml, preferably 100 mg/ml to 165 mg/ml, of an anti-human PD-1 antibody, preferably of pembrolizumab; trehalose dihydrate; a non-ionic surfactant and citrate buffer, pH 5.0 to 5.8.

In one embodiment, the present invention relates to a liquid pharmaceutical composition comprising: 90 mg/ml to 350 mg/ml, preferably 100 mg/ml to 165 mg/ml, of an anti-human PD-1 antibody, preferably of pembrolizumab; trehalose dihydrate; polysorbate 80 and citrate buffer, pH 5.0 to 5.8.

In one embodiment, the present invention relates to a liquid pharmaceutical composition comprising: 90 mg/ml to 350 mg/ml, preferably 100 mg/ml to 165 mg/ml, of an anti-human PD-1 antibody, preferably of pembrolizumab; trehalose dihydrate; a non-ionic surfactant and 2 mM citrate buffer, pH 5.0 to 5.8.

In one embodiment, the present invention relates to a liquid pharmaceutical composition comprising: 90 mg/ml to 350 mg/ml, preferably 100 mg/ml to 165 mg/ml, of an anti-human PD-1 antibody, preferably of pembrolizumab; trehalose dihydrate; polysorbate 80 and 2 mM citrate buffer, pH 5.0 to 5.8.

In one embodiment, the liquid pharmaceutical composition comprising 90 mg/ml to 350 mg/ml, preferably 100 mg/ml to 165 mg/ml, of an anti-human PD-1 antibody, preferably of pembrolizumab, may additionally contain arginine. If arginine is present, it may be present in a concentration of 10 mM to 200 mM. If arginine is present in the liquid pharmaceutical composition comprising 90 mg/ml to 350 mg/ml, preferably 100 mg/ml to 165 mg/ml, of an anti-human PD-1 antibody, preferably of pembrolizumab, the concentration of trehalose may be reduced to adjust the osmolality.

In one embodiment, the present invention relates to a liquid pharmaceutical composition comprising: 90 mg/ml to 350 mg/ml, preferably 100 mg/ml to 165 mg/ml, of an anti-human PD-1 antibody, preferably of pembrolizumab; arginine; polysorbate 80 and 2 mM citrate buffer, pH 5.0 to 5.8.

In one embodiment, the present invention relates to a liquid pharmaceutical composition comprising: 80 mg/ml to 180 mg/ml of an anti-human PD1 antibody; 150 mM to 250 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 80 mg/ml to 180 mg/ml of an anti-human PD1 antibody; 150 mM to 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 80 mg/ml to 180 mg/ml of an anti-human PD1 antibody; 150 mM or 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 80 mg/ml to 180 mg/ml of an anti-human PD1 antibody; 150 mM to 250 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 80 mg/ml to 180 mg/ml of an anti-human PD1 antibody; 150 mM to 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 80 mg/ml to 180 mg/ml of an anti-human PD1 antibody; 150 mM or 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 80 mg/ml to 180 mg/ml of an anti-human PD1 antibody; 150 mM to 250 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 80 mg/ml to 180 mg/ml of an anti-human PD1 antibody; 150 mM to 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 80 mg/ml to 180 mg/ml of an anti-human PD1 antibody; 150 mM or 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 80 mg/ml to 180 mg/ml of an anti-human PD1 antibody; 150 mM to 250 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 80 mg/ml to 180 mg/ml of an anti-human PD1 antibody; 150 mM to 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 80 mg/ml to 180 mg/ml of an anti-human PD1 antibody; 150 mM or 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 80 mg/ml to 180 mg/ml of pembrolizumab; 150 mM to 250 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 80 mg/ml to 180 mg/ml of pembrolizumab; 150 mM to 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 80 mg/ml to 180 mg/ml of pembrolizumab; 150 mM or 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 80 mg/ml to 180 mg/ml of pembrolizumab; 150 mM to 250 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 80 mg/ml to 180 mg/ml of pembrolizumab; 150 mM to 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 80 mg/ml to 180 mg/ml of pembrolizumab; 150 mM or 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 80 mg/ml to 180 mg/ml of pembrolizumab; 150 mM to 250 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 80 mg/ml to 180 mg/ml of pembrolizumab; 150 mM to 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 80 mg/ml to 180 mg/ml of pembrolizumab; 150 mM or 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 80 mg/ml to 180 mg/ml of pembrolizumab; 150 mM to 250 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 80 mg/ml to 180 mg/ml of pembrolizumab; 150 mM to 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 80 mg/ml to 180 mg/ml of pembrolizumab; 150 mM or 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 100, 150 or 165 mg/ml of an anti-human PD1 antibody; 150 mM to 250 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 100, 150 or 165 mg/ml of an anti-human PD1 antibody; 150 mM to 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 100, 150 or 165 mg/ml of an anti-human PD1 antibody; 150 mM or 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 100, 150 or 165 mg/ml of an anti-human PD1 antibody; 150 mM to 250 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 100, 150 or 165 mg/ml of an anti-human PD1 antibody; 150 mM to 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 100, 150 or 165 mg/ml of an anti-human PD1 antibody; 150 mM or 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 100, 150 or 165 mg/ml of an anti-human PD1 antibody; 150 mM to 250 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 100, 150 or 165 mg/ml of an anti-human PD1 antibody; 150 mM to 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 100, 150 or 165 mg/ml of an anti-human PD1 antibody; 150 mM or 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 100, 150 or 165 mg/ml of an anti-human PD1 antibody; 150 mM to 250 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 100, 150 or 165 mg/ml of an anti-human PD1 antibody; 150 mM to 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 100, 150 or 165 mg/ml of an anti-human PD1 antibody; 150 mM or 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 100, 150 or 165 mg/ml of pembrolizumab; 150 mM to 250 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 100, 150 or 165 mg/ml of pembrolizumab; 150 mM to 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 100, 150 or 165 mg/ml of pembrolizumab; 150 mM or 205 mM of trehalose dihydrate; 0.1 mg/ml to 0.4 mg/ml of a non-ionic surfactant and 1 to 50 mM of citrate buffer, pH 5.0 to 5.8.

In one embodiment, the present invention relates to a liquid pharmaceutical composition comprising: 100, 150 or 165 mg/ml of pembrolizumab; 150 mM to 250 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 100, 150 or 165 mg/ml of pembrolizumab; 150 mM to 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 100, 150 or 165 mg/ml of pembrolizumab; 150 mM or 205 mM of trehalose dihydrate; 0.1 mg/ml to 0.4 mg/ml of polysorbate 80 and 1 to 50 mM of citrate buffer, pH 5.0 to 5.8.

In one embodiment, the present invention relates to a liquid pharmaceutical composition comprising: 100, 150 or 165 mg/ml of pembrolizumab; 150 mM to 250 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 100, 150 or 165 mg/ml of pembrolizumab; 150 mM to 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 100, 150 or 165 mg/ml of pembrolizumab; 150 mM or 205 mM of trehalose dihydrate; 0.1 mg/ml to 0.4 mg/ml of a non-ionic surfactant and 1 to 50 mM of citrate buffer, pH 5.5.

In one embodiment, the present invention relates to a liquid pharmaceutical composition comprising: 100, 150 or 165 mg/ml of pembrolizumab; 150 mM to 250 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 100, 150 or 165 mg/ml of pembrolizumab; 150 mM to 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 100, 150 or 165 mg/ml of pembrolizumab; 150 mM or 205 mM of trehalose dihydrate; 0.1 mg/ml to 0.4 mg/ml of polysorbate 80 and 1 to 50 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.2 mg/ml of a non-ionic surfactant and 1 to 50 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.2 mg/ml of a non-ionic surfactant and 1 to 50 mM of citrate buffer, pH 5.0 to 5.8.

In one embodiment, the present invention relates to a liquid pharmaceutical composition comprising: 80 to 180 mg/ml of an anti-human PD1 antibody; 150 mM or 205 mM of trehalose dihydrate; 0.2 mg/ml of a non-ionic surfactant and 1 to 50 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.2 mg/ml of polysorbate 80 and 1 to 50 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.2 mg/ml of polysorbate 80 and 1 to 50 mM of citrate buffer, pH 5.0 to 5.8.

In one embodiment, the present invention relates to a liquid pharmaceutical composition comprising: 80 to 180 mg/ml of an anti-human PD1 antibody; 150 mM or 205 mM of trehalose dihydrate; 0.2 mg/ml of polysorbate 80 and 1 to 50 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.2 mg/ml of a non-ionic surfactant and 1 to 50 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.2 mg/ml of a non-ionic surfactant and 1 to 50 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.2 mg/ml of a non-ionic surfactant and 1 to 50 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.2 mg/ml of polysorbate 80 and 1 to 50 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.2 mg/ml of polysorbate 80 and 1 to 50 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.2 mg/ml of polysorbate 80 and 1 to 50 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.2 mg/ml of a non-ionic surfactant and 1 to 50 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.2 mg/ml of a non-ionic surfactant and 1 to 50 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.2 mg/ml of a non-ionic surfactant and 1 to 50 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 0.2 mg/ml of polysorbate 80 and 1 to 50 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.2 mg/ml of polysorbate 80 and 1 to 50 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.2 mg/ml of polysorbate 80 and 1 to 50 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 0.2 mg/ml of a non-ionic surfactant and 1 to 50 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.2 mg/ml of a non-ionic surfactant and 1 to 50 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.2 mg/ml of a non-ionic surfactant and 1 to 50 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.2 mg/ml of polysorbate 80 and 1 to 50 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.2 mg/ml of polysorbate 80 and 1 to 50 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.2 mg/ml of polysorbate 80 and 1 to 50 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of a non-ionic surfactant and 2 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of a non-ionic surfactant and 2 mM of citrate buffer, pH 5.0 to 5.8.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of a non-ionic surfactant and 2 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of polysorbate 80 and 2 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of polysorbate 80 and 2 mM of citrate buffer, pH 5.0 to 5.8.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of polysorbate 80 and 2 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of a non-ionic surfactant and 2 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of a non-ionic surfactant and 2 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of a non-ionic surfactant and 2 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of a non-ionic surfactant and 5 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of a non-ionic surfactant and 5 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of a non-ionic surfactant and 5 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of a non-ionic surfactant and 10 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of a non-ionic surfactant and 10 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of a non-ionic surfactant and 10 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of polysorbate 80 and 2 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of polysorbate 80 and 2 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of polysorbate 80 and 2 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of polysorbate 80 and 5 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of polysorbate 80 and 5 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of polysorbate 80 and 5 mM of citrate buffer, pH 5.5.

In one embodiment, the present invention relates to a liquid pharmaceutical composition comprising: 80 to 180 mg/ml of an anti-human PD1 antibody; 150 mM to 250 mM of trehalose dihydrate; 0.1 mg/ml to 0.4 mg/ml of polysorbate 80 and 10 mM of citrate buffer, pH 5.5.

In one embodiment, the present invention relates to a liquid pharmaceutical composition comprising: 80 to 180 mg/ml of an anti-human PD1 antibody; 150 mM to 205 mM of trehalose dihydrate; 0.1 mg/ml to 0.4 mg/ml of polysorbate 80 and 10 mM of citrate buffer, pH 5.5.

In one embodiment, the present invention relates to a liquid pharmaceutical composition comprising: 80 to 180 mg/ml of an anti-human PD1 antibody; 150 mM or 205 mM of trehalose dihydrate; 0.1 mg/ml to 0.4 mg/ml of polysorbate 80 and 10 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of a non-ionic surfactant and 2 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of a non-ionic surfactant and 2 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of a non-ionic surfactant and 2 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of a non-ionic surfactant and 5 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 100 mM to 350 mM of arginine; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of a non-ionic surfactant and 10 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of polysorbate 80 and 2 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of polysorbate 80 and 2 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of polysorbate 80 and 2 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of polysorbate 80 and 5 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of polysorbate 80 and 10 mM of citrate buffer, pH 5.0 to 5.8.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of a non-ionic surfactant and 2 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of a non-ionic surfactant and 2 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of a non-ionic surfactant and 2 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of a non-ionic surfactant and 5 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of a non-ionic surfactant and 5 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of a non-ionic surfactant and 5 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of a non-ionic surfactant and 10 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of a non-ionic surfactant and 10 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of a non-ionic surfactant and 10 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of polysorbate 80 and 2 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of polysorbate 80 and 2 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of polysorbate 80 and 2 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of polysorbate 80 and 5 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of polysorbate 80 and 5 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of polysorbate 80 and 5 mM of citrate buffer, pH 5.5.

In one embodiment, the present invention relates to a liquid pharmaceutical composition comprising: 80 to 180 mg/ml of pembrolizumab; 150 mM to 250 mM of trehalose dihydrate; 0.1 mg/ml to 0.4 mg/ml of polysorbate 80 and 10 mM of citrate buffer, pH 5.5.

In one embodiment, the present invention relates to a liquid pharmaceutical composition comprising: 80 to 180 mg/ml of pembrolizumab; 150 mM to 205 mM of trehalose dihydrate; 0.1 mg/ml to 0.4 mg/ml of polysorbate 80 and 10 mM of citrate buffer, pH 5.5.

In one embodiment, the present invention relates to a liquid pharmaceutical composition comprising: 80 to 180 mg/ml of pembrolizumab; 150 mM or 205 mM of trehalose dihydrate; 0.1 mg/ml to 0.4 mg/ml of polysorbate 80 and 10 mM of citrate buffer, pH 5.5.

In one embodiment, the present invention relates to a liquid pharmaceutical composition comprising: 100 mg/ml, 150 mg/ml or 165 mg/ml of an anti-PD1 antibody; 205 mM trehalose dihydrate; 0.2 mg/ml polysorbate 80 and 3, 5 or 10 mM of citrate buffer, pH 5.0 to 5.8.

In one embodiment, the present invention relates to a liquid pharmaceutical composition comprising: 100 mg/ml, 150 mg/ml or 165 mg/ml of an anti-PD1 antibody; 205 mM trehalose dihydrate; 0.2 mg/ml polysorbate 80 and 3, 5 or 10 mM of citrate buffer, pH 5.5.

In one embodiment, the present invention relates to a liquid pharmaceutical composition comprising: 100 mg/ml, 150 mg/ml or 165 mg/ml of pembrolizumab; 205 mM trehalose dihydrate; 0.2 mg/ml polysorbate 80 and 5 or 10 mM of citrate buffer, pH 5.0 to 5.8.

In one embodiment, the present invention relates to a liquid pharmaceutical composition comprising: 100 mg/ml, 150 mg/ml or 165 mg/ml of pembrolizumab; 205 mM trehalose dihydrate; 0.2 mg/ml polysorbate 80 and 5 or 10 mM of citrate buffer, pH 5.5.

In one embodiment, the present invention relates to a liquid pharmaceutical composition comprising: 100 mg/ml, 150 mg/ml or 165 mg/ml of an anti-PD1 antibody; 150 mM trehalose dihydrate; 0.2 mg/ml polysorbate 80 and 3, 5 or 10 mM of citrate buffer, pH 5.0 to 5.8.

In one embodiment, the present invention relates to a liquid pharmaceutical composition comprising: 100 mg/ml, 150 mg/ml or 165 mg/ml of an anti-PD1 antibody; 150 mM trehalose dihydrate; 0.2 mg/ml polysorbate 80 and 3, 5 or 10 mM of citrate buffer, pH 5.5.

In one embodiment, the present invention relates to a liquid pharmaceutical composition comprising: 100 mg/ml, 150 mg/ml or 165 mg/ml of pembrolizumab; 150 mM trehalose dihydrate; 0.2 mg/ml polysorbate 80 and 5 or 10 mM of citrate buffer, pH 5.0 to 5.8.

In one embodiment, the present invention relates to a liquid pharmaceutical composition comprising: 100 mg/ml, 150 mg/ml or 165 mg/ml of pembrolizumab; 150 mM trehalose dihydrate; 0.2 mg/ml polysorbate 80 and 5 or 10 mM of citrate buffer, pH 5.5.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

The detailed description is merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention.

### EXAMPLES

1. Formulation Development of 25 mg/mL Pembrolizumab Formulations

### Example 1: Screening of Pembrolizumab Formulations

### 1. Sample Preparation

Two independent design of experiment studies (DoE) were performed in order to screen for suitable pembrolizumab formulations.

First a DoE study (including formulations No. (1)-(32) of Table 1) was prepared and evaluated in a D-optimal design for obtaining linear and quadratic effects of components, as well as selected interactions. As single components histidine (covering the concentration range 0-30 mM), citrate (0-30 mM), trehalose dihydrate (0-300 mM), mannitol (0-300 mM), polysorbate 20 (0-0.2 mg/mL) and pH (5.0-6.5) were evaluated for their stabilizing effects. Center point (No. (7)) was included in the study as triplicate preparation.

A second DoE study (including formulations No. (33)-(55) of Table 1) was prepared and evaluated also in a D-optimal design for obtaining linear and quadratic effects of components, as well as selected interactions. As single components lysine HCl (covering the concentration range 0-150 mM), arginine HCl (0-150 mM), proline (0-100 mM), glycine (0-75 mM) and glycerol (0-200 mM) were evaluated for their stabilizing effects. Core components buffer (10 mM citrate, pH 5.5) and 0.2 mg/mL polysorbate 20 were kept constant in all formulations. Center point (No. (39)) was included in the study as duplicate preparation.

Pembrolizumab at 25 mg/mL was used as starting material and the 57 formulations listed in Table 1 were prepared via a 3-step-dialysis. Dialyzed pembrolizumab was adjusted to 25 mg/mL±20% and 0.22 μm sterile filtered. The formulations shown in Table 1 were initially analyzed for protein concentration by UV-VIS spectroscopy at 280 nm and correct pH adjustment before starting an accelerated aging stability program. Storage conditions are shown in Table 2. In brief, samples were placed on storage for up to 2 weeks at 40° C./75% relative humidity (RH). In addition, samples underwent two conditions of mechanical stressing (overhead rotation, orbital shaking) as well as five freeze/thaw cycles (−80° C./+25° C.) Pembrolizumab in the Keytruda® labelled formulation buffer was included in the study as control sample (57) and dialyzed head-to-head with the other formulations shown in Table 1.

TABLE 1
Detailed information on formulations prepared within this study (example 1)

| No. | Pembrolizumab | Buffer system | Stabilizer | Surfactant | pH |
|---|---|---|---|---|---|
| (1) | 25 mg/mL | 15 mM histidine/histidine HCl, | n.a. | 0.2 mg/mL polysorbate 20 | 5.0 |
| | | 30 mM citric acid/sodium citrate | | | |
| (2) | 25 mg/mL | 15 mM citric acid/sodium citrate | 300 mM mannitol | n.a. | 5.8 |
| (3) | 25 mg/mL | 30 mM histidine/histidine HCl, | 300 mM trehalose dihydrate, | n.a. | 6.2 |
| | | 30 mM citric acid/sodium citrate | 300 mM mannitol | | |
| (4) | 25 mg/mL | 30 mM histidine/histidine HCl | 150 mM mannitol | n.a. | 5.0 |
| (5) | 25 mg/mL | 30 mM citric acid/sodium citrate | 150 mM trehalose dihydrate, | 0.2 mg/mL polysorbate 20 | 6.2 |
| | | | 300 mM mannitol | | |
| (6) | 25 mg/mL | 30 mM histidine/histidine HCl, | n.a. | n.a | 6.2 |
| | | 30 mM citric acid/sodium citrate | | | |
| (7) | 25 mg/mL | 15 mM histidine/histidine HCl, | 150 mM trehalose dihydrate, | 0.1 mg/mL polysorbate 20 | 5.6 |
| | | 15 mM citric acid/sodium citrate | 150 mM mannitol | | |
| (8) | 25 mg/mL | 30 mM histidine/histidine HCl | 225 mM trehalose dihydrate, | n.a. | 5.0 |
| | | | 300 mM mannitol | | |
| (9) | 25 mg/mL | 30 mM citric acid/sodium citrate | 300 mM trehalose dihydrate | 0.2 mg/mL polysorbate 20 | 5.8 |
| (10) | 25 mg/mL | 30 mM citric acid/sodium citrate | 300 mM trehalose dihydrate, | n.a. | 5.0 |
| | | | 150 mM mannitol | | |
| (11) | 25 mg/mL | 30 mM citric acid/sodium citrate | 75 mM mannitol | 0.2 mg/mL polysorbate 20 | 6.2 |
| (12) | 25 mg/mL | 15 mM histidine/histidine HCl, | 300 mM trehalose dihydrate, | 0.2 mg/mL polysorbate 20 | 5.0 |
| | | 15 mM citric acid/sodium citrate | 300 mM mannitol | | |
| (13) | 25 mg/mL | 30 mM histidine/histidine HCl | 75 mM trehalose dihydrate | 0.2 mg/mL polysorbate 20 | 6.2 |
| (14) | 25 mg/mL | 30 mM histidine/histidine HCl | 300 mM trehalose dihydrate, | 0.2 mg/mL polysorbate 20 | 6.2 |
| | | | 225 mM mannitol | | |
| (15) | 25 mg/mL | 30 mM histidine/histidine HCl, | 300 mM trehalose dihydrate | 0.1 mg/mL polysorbate 20 | 5.0 |
| | | 30 mM citric acid/sodium citrate | | | |
| (16) | 25 mg/mL | 15 mM citric acid/sodium citrate | 300 mM trehalose dihydrate | 0.1 mg/mL polysorbate 20 | 6.2 |
| (17) | 25 mg/mL | 30 mM histidine/histidine HCl | 300 mM trehalose dihydrate | n.a. | 5.4 |
| (18) | 25 mg/mL | 15 mM citric acid/sodium citrate | 150 mM trehalose dihydrate | n.a. | 5.0 |
| (19) | 25 mg/mL | 7.5 mM histidine/histidine HCl | 300 mM mannitol | 0.2 mg/mL polysorbate 20 | 5.0 |
| (20) | 25 mg/mL | 7.5 mM histidine/histidine HCl | 300 mM trehalose dihydrate | 0.15 mg/mL polysorbate 20 | 5.0 |
| (21) | 25 mg/mL | 7.5 mM histidine/histidine HCl | n.a. | n.a. | 6.2 |
| (22) | 25 mg/mL | 30 mM histidine/histidine HCl | 300 mM mannitol | 0.1 mg/mL polysorbate 20 | 6.2 |
| (23) | 25 mg/mL | 7.5 mM histidine/histidine HCl | 300 mM trehalose dihydrate, | n.a. | 6.2 |
| | | | 300 mM mannitol | | |
| (24) | 25 mg/mL | 30 mM citric acid/sodium citrate | 300 mM mannitol | 0.05 mg/mL polysorbate 20 | 5.0 |
| (25) | 25 mg/mL | 15 mM histidine/histidine HCl, | 150 mM trehalose dihydrate, | 0.1 mg/mL polysorbate 20 | 5.8 |
| | | 30 mM citric acid/sodium citrate | 150 mM mannitol | | |
| (26) | 25 mg/mL | 30 mM histidine/histidine HCl, | 300 mM mannitol | 0.2 mg/mL polysorbate 20 | 5.0 |
| | | 30 mM citric acid/sodium citrate | | | |
| (27) | 25 mg/mL | 10 mM histidine/histidine HCl, | 205 mM trehalose dihydrate | 0.2 mg/mL polysorbate 20 | 5.5 |
| | | 10 mM citric acid/sodium citrate | | | |
| (28) | 25 mg/mL | 10 mM citric acid/sodium citrate | 205 mM sucrose | 0.1 mg/mL polysorbate 20 | 5.5 |
| (29) | 25 mg/mL | 20 mM citric acid/sodium citrate | 205 mM trehalose dihydrate | 0.2 mg/mL polysorbate 20 | 5.5 |
| (30) | 25 mg/mL | 20 mM citric acid/sodium citrate | 102.5 mM trehalose dihydrate, | 0.2 mg/mL polysorbate 20 | 5.5 |
| | | | 102.5 mM mannitol | | |
| (31) | 25 mg/mL | 10 mM citric acid/sodium citrate | 205 mM trehalose dihydrate | 0.1 mg/mL polysorbate 20 | 5.5 |
| (32) | 25 mg/mL | 10 mM histidine/histidine HCl, | 102.5 mM mannitol, | 0.2 mg/mL polysorbate 20 | 5.5 |
| | | 10 mM citric acid/sodium citrate | 102.5 mM sucrose | | |
| (33) | 25 mg/mL | 10 mM citric acid/sodium citrate | 75 mM lysine/lysine HCl, | 0.2 mg/mL polysorbate 20 | 5.5 |
| | | | 75 mM arginine/arginine HCl, | | |
| | | | 50 mM proline, 75 mM glycine, | | |
| | | | 200 mM glycerol | | |
| (34) | 25 mg/mL | 10 mM citric acid/sodium citrate | 100 mM proline, 75 mM glycine | 0.2 mg/mL polysorbate 20 | 5.5 |
| (35) | 25 mg/mL | 10 mM citric acid/sodium citrate | 37.5 mM arginine/arginine HCl | 0.2 mg/mL polysorbate 20 | 5.5 |
| (36) | 25 mg/mL | 10 mM citric acid/sodium citrate | 150 mM arginine/arginine HCl, | 0.2 mg/mL polysorbate 20 | 5.5 |
| | | | 100 mM proline, 75 mM glycine, | | |
| | | | 200 mM glycerol | | |
| (37) | 25 mg/mL | 10 mM citric acid/sodium citrate | 150 mM arginine/arginine HCl, | 0.2 mg/mL polysorbate 20 | 5.5 |
| | | | 200 mM glycerol | | |
| (38) | 25 mg/mL | 10 mM citric acid/sodium citrate | 150 mM arginine/arginine HCl, | 0.2 mg/mL polysorbate 20 | 5.5 |
| | | | 75 mM glycine | | |
| (39) | 25 mg/mL | 10 mM citric acid/sodium citrate | 75 mM lysine/lysine HCl, | 0.2 mg/mL polysorbate 20 | 5.5 |
| | | | 75 mM arginine/arginine HCl, | | |
| | | | 50 mM proline, 37.5 mM glycine, | | |
| | | | 100 mM glycerol | | |
| (40) | 25 mg/mL | 10 mM citric acid/sodium citrate | 150 mM lysine/lysine HCl, | 0.2 mg/mL polysorbate 20 | 5.5 |
| | | | 100 mM proline | | |
| (41) | 25 mg/mL | 10 mM citric acid/sodium citrate | 150 mM lysine/lysine HCl, | 0.2 mg/mL polysorbate 20 | 5.5 |
| | | | 150 mM arginine/arginine HCl, | | |
| | | | 75 mM glycine, 200 mM glycerol | | |
| (42) | 25 mg/mL | 10 mM citric acid/sodium citrate | 150 mM lysine/lysine HCl, | 0.2 mg/mL polysorbate 20 | 5.5 |
| | | | 150 mM arginine/arginine HCl, | | |
| | | | 100 mM proline, 75 mM glycine | | |
| (43) | 25 mg/mL | 10 mM citric acid/sodium citrate | 150 mM lysine/lysine HCl, | 0.2 mg/mL polysorbate 20 | 5.5 |
| | | | 75 mM glycine | | |
| (44) | 25 mg/mL | 10 mM citric acid/sodium citrate | 50 mM proline, 37.5 mM glycine | 0.2 mg/mL polysorbate 20 | 5.5 |
| (45) | 25 mg/mL | 10 mM citric acid/sodium citrate | 150 mM lysine/lysine HCl, | 0.2 mg/mL polysorbate 20 | 5.5 |
| | | | 150 mM arginine/arginine HCl, | | |
| | | | 100 mM proline, 200 mM glycerol | | |
| (46) | 25 mg/mL | 10 mM citric acid/sodium citrate | 150 mM lysine/lysine HCl, | 0.2 mg/mL polysorbate 20 | 5.5 |
| | | | 200 mM glycerol | | |
| (47) | 25 mg/mL | 10 mM citric acid/sodium citrate | 75 mM lysine/lysine HCl, | 0.2 mg/mL polysorbate 20 | 5.5 |
| | | | 150 mM arginine/arginine HCl, | | |
| | | | 37.5 mM glycine | | |
| (48) | 25 mg/mL | 10 mM citric acid/sodium citrate | 150 mM lysine/lysine HCl, | 0.2 mg/mL polysorbate 20 | 5.5 |
| | | | 75 mM arginine/arginine HCl, | | |
| | | | 100 mM proline, 37.5 mM glycine, | | |
| | | | 100 mM glycerol | | |
| (49) | 25 mg/mL | 10 mM citric acid/sodium citrate | 100 mM proline, 200 mM glycerol | 0.2 mg/mL polysorbate 20 | 5.5 |
| (50) | 25 mg/mL | 10 mM citric acid/sodium citrate | 150 mM lysine/lysine HCl, | 0.2 mg/mL polysorbate 20 | 5.5 |
| | | | 100 mM proline, 75 mM glycine, | | |
| | | | 200 mM glycerol | | |
| (51) | 25 mg/mL | 10 mM citric acid/sodium citrate | 150 mM arginine/arginine HCl, | 0.2 mg/mL polysorbate 20 | 5.5 |
| | | | 100 mM proline | | |
| (52) | 25 mg/mL | 10 mM citric acid/sodium citrate | 75 mM lysine/lysine HCl, | 0.2 mg/mL polysorbate 20 | 5.5 |
| | | | 100 mM glycerol | | |
| (53) | 25 mg/mL | 10 mM citric acid/sodium citrate | 150 mM arginine/arginine HCl, | 0.2 mg/mL polysorbate 20 | 5.5 |
| | | | 50 mM proline, 100 mM glycerol | | |
| (54) | 25 mg/mL | 10 mM citric acid/sodium citrate | 150 mM lysine/lysine HCl, | 0.2 mg/mL polysorbate 20 | 5.5 |
| | | | 150 mM arginine/arginine HCl | | |
| (55) | 25 mg/mL | 10 mM citric acid/sodium citrate | 75 mM glycine, 200 mM glycerol | 0.2 mg/mL polysorbate 20 | 5.5 |
| (56) | 25 mg/mL | 10 mM citric acid/sodium citrate | 75 mM lysine/lysine HCl, | 0.2 mg/mL Poloxamer 188 | 5.5 |
| | | | 75 mM arginine/arginine HCl, | | |
| | | | 50 mM proline, 37.5 mM glycine, | | |
| | | | 100 mM glycerol | | |
| (57) | 25 mg/mL | 10 mM histidine/histidine HCl | 205 mM sucrose | 0.2 mg/mL polysorbate 80 | 5.5 |

TABLE 2
Accelerated aging of pembrolizumab formulations

| | Time Point | | |
|---|---|---|---|
| Stress Type | t0 | 1 week | 2 weeks |
| 40° C. ± 2° C./75% RH ± 5% RH | X | X | X |

| | |
|---|---|
| Mechanical stressing (up to 24 h) | 2 conditions (orbital shaking |
| | and overhead rotation) |
| Freeze/thaw (−80° C./+25° C.) | 5 cycles |

Samples were analyzed by size exclusion chromatography (SE-HPLC) for the presence of high molecular weight species (HMWS). In addition, ion exchange chromatography (IEX-HPLC) was used to detect modifications leading to charge heterogeneities.

### 2. Analysis of Protein Content by UV-VIS and Determination of pH and Osmolality

All formulations prepared according to Table 1 were analyzed for their protein content as well as for pH and osmolality before starting the stability study (t0). All samples met the acceptance criteria for protein content (25 mg/mL±20% pembrolizumab) and pH (target±0.2). No visible particles or changes in color were detected in all samples.

### 3. Analysis of High Molecular Weight Species (HMWS) by SE-HPLC

The samples of the accelerated aging study were diluted in 20 mM histidine pH 5.5 to a final concentration of 1 mg/mL and 5 μL thereof were injected to a TSKgel UP-SW3000 (Tosoh, 4.6×150 mm, 2 μm) column to detect high molecular weight species (HMWS) of pembrolizumab.

The protein was eluted by isocratic elution using 0.1 M sodium phosphate buffer with 0.05% (v/v) sodium azide (pH 6.7) at a flow rate of 0.35 mL/min at 25° C. Eluted species were detected at a wavelength of 280 nm and displayed on a graph showing the concentration of the eluted species versus time. The elution profile showed a monomer peak with the non-aggregated protein and peaks of the protein representing higher molecular weight species (HMWS) of the protein. The areas of all peaks were determined.

Table 3 summarizes the percentage of peak area for the HMWS in relation to the total peak area of the eluted species for the formulations shown in Table 1. Each sample was measured in triplicates.

TABLE 3
Overview of HMWS determined using SE-HPLC

| | | HMWS [%] |
|---|---|---|
| | | Mean of three |
| Condition | Formulation | measurements |
| t0 | (1) | 0.7 |
| | (2) | 0.8 |
| | (3) | 0.7 |
| | (4) | 0.7 |
| | (5) | 0.8 |
| | (6) | 0.7 |
| | (7) | 0.7 |
| | (8) | 0.7 |
| | (9) | 0.7 |
| | (10) | 0.7 |
| | (11) | 0.8 |
| | (12) | 0.7 |
| | (13) | 0.8 |
| | (14) | 0.7 |
| | (15) | 0.7 |
| | (16) | 0.8 |
| | (17) | 0.7 |
| | (18) | 0.7 |
| | (19) | 0.7 |
| | (20) | 0.7 |
| | (21) | 0.7 |
| | (22) | 0.7 |
| | (23) | 0.7 |
| | (24) | 0.7 |
| | (25) | 0.7 |
| | (26) | 0.7 |
| | (27) | 0.7 |
| | (28) | 0.7 |
| | (29) | 0.7 |
| | (30) | 0.7 |
| | (31) | 0.7 |
| | (32) | 0.7 |
| | (33) | 0.7 |
| | (34) | 0.7 |
| | (35) | 0.7 |
| | (36) | 0.7 |
| | (37) | 0.7 |
| | (38) | 0.7 |
| | (39) | 0.7 |
| | (40) | 0.7 |
| | (41) | 0.7 |
| | (42) | 0.7 |
| | (43) | 0.7 |
| | (44) | 0.7 |
| | (45) | 0.7 |
| | (46) | 0.7 |
| | (47) | 0.7 |
| | (48) | 0.7 |
| | (49) | 0.7 |
| | (50) | 0.7 |
| | (51) | 0.7 |
| | (52) | 0.8 |
| | (53) | 0.7 |
| | (54) | 0.7 |
| | (55) | 0.8 |
| | (56) | 0.7 |
| | (57) | 0.7 |
| 2 weeks | (1) | 1.0 |
| 40° C. | (2) | 1.2 |
| | (3) | 0.9 |
| | (4) | 0.9 |
| | (5) | 1.3 |
| | (6) | 1.0 |
| | (7) | 0.9 |
| | (8) | 0.8 |
| | (9) | 1.3 |
| | (10) | 1.0 |
| | (11) | 1.4 |
| | (12) | 0.9 |
| | (13) | 1.0 |
| | (14) | 0.9 |
| | (15) | 1.0 |
| | (16) | 1.4 |
| | (17) | 0.8 |
| | (18) | 1.1 |
| | (19) | 0.8 |
| | (20) | 0.9 |
| | (21) | 1.1 |
| | (22) | 1.0 |
| | (23) | 1.0 |
| | (24) | 1.1 |
| | (25) | 1.0 |
| | (26) | 0.9 |
| | (27) | 1.0 |
| | (28) | 1.2 |
| | (29) | 1.1 |
| | (30) | 1.2 |
| | (31) | 1.2 |
| | (32) | 1.0 |
| | (33) | 0.9 |
| | (34) | 1.2 |
| | (35) | 1.2 |
| | (36) | 0.9 |
| | (37) | 1.0 |
| | (38) | 1.0 |
| | (39) | 1.0 |
| | (40) | 1.0 |
| | (41) | 0.9 |
| | (42) | 0.9 |
| | (43) | 1.0 |
| | (44) | 1.3 |
| | (45) | 0.9 |
| | (46) | 1.1 |
| | (47) | 1.0 |
| | (48) | 1.0 |
| | (49) | 1.2 |
| | (50) | 1.0 |
| | (51) | 1.0 |
| | (52) | 1.1 |
| | (53) | 1.0 |
| | (54) | 1.0 |
| | (55) | 1.2 |
| | (56) | 1.0 |
| | (57) | 0.9 |

After storage for two weeks at 40° C., the HMWS levels increased slightly to 0.8% to 1.4% compared to t0 (0.7%) for all formulations tested within the design of experiment setup. For formulations (1) to (32) the effect of the buffer can be summarized as follows: histidine has an optimum at a molarity of 20-25 mM and a pH of 5.2-5.8.

For citrate, the pH optimum shifts to slightly lower pH values (ca. pH 5.0-5.6) and a slight destabilizing effect was observed at elevated temperatures for high concentrations of citrate, which decreased with decreasing citrate molarity. Hence, histidine as well as citrate (especially when used at low concentrations up to 10 mM citrate) are optimal buffering systems for pembrolizumab.

For formulations (33) to (56) citrate buffer was used as a core component.

The concentration of the sugar and the sugar alcohol had only minor effects on the HMWS levels in formulations (1) to (32), as the formulations were robust in the tested concentration range of sugars. Comparing formulations (33) through (56), which contained amino acids and/or glycerol as stabilizing excipients, optimal conditions for formulations of pembrolizumab were identified in samples (33), (36), (41), (42) and (45). Further evaluation revealed that with a combination of high concentrations of arginine and/or lysine and 100 mM glycerol comparable results to the sugar and/or sugar alcohol used in formulations (1) to (32) could be achieved in terms of HMWS levels. The formulation containing Poloxamer 188 (56) yielded results comparable to polysorbate 20 (PS20)-containing formulations as well as to the control formulation (57), which contained PS80.

All tested formulations except for formulations (2), (4), (19), (22) and (26) remained stable after multiple freeze/thaw cycles. These formulations contained high concentrations of mannitol as the only stabilizing excipient and showed a severe increase in aggregation after freezing.

After storage for one week at 40° C. as well as after applying mechanical stressing by orbital shaking and overhead rotation the same trends as described above were observed for formulations (1) to (57).

### 4. Detection of Acidic and Basic Species by IEX-HPLC

The samples of the accelerated aging study were diluted in eluent A (20 mM MES, pH 6.2) to a final concentration of 1 mg/mL and 30 μL thereof were injected to a MabPac SCX-10 (Thermo Scientific, 4×250 mm, 10 μm) column in order to detect modifications of pembrolizumab leading to charge heterogeneities. Proteins were eluted using a mixture of mobile phase A (20 mM MES, pH 6.2) and B (20 mM MES, 120 mM KCl, pH 6.2) at a flow rate of 1 mL/min at 40° C. by applying a salt gradient (KCl).

Eluted species were detected at a wavelength of 280 nm and displayed on a graph showing the concentration of the eluted species versus time. The elution profile showed a main peak as well as several peaks representing the acidic and basic variants. The areas of all peaks were determined.

Table 4 summarizes the percentage of the peak area for the acidic species in relation to the total peak area of the eluted species for the formulations shown in Table 1. Each sample was measured in triplicates.

TABLE 4
Overview of basic species determined using IEX-HPLC

| | | Acidic species [%] |
|---|---|---|
| | | Mean of three |
| Condition | Formulation | measurements |
| t0 | (1) | 16.9 |
| | (2) | 16.7 |
| | (3) | 17.0 |
| | (4) | 16.9 |
| | (5) | 17.0 |
| | (6) | 17.2 |
| | (7) | 16.9 |
| | (8) | 17.0 |
| | (9) | 17.2 |
| | (10) | 17.2 |
| | (11) | 17.1 |
| | (12) | 17.2 |
| | (13) | 17.4 |
| | (14) | 17.2 |
| | (15) | 17.4 |
| | (16) | 17.2 |
| | (17) | 17.1 |
| | (18) | 17.2 |
| | (19) | 17.1 |
| | (20) | 17.1 |
| | (21) | 17.2 |
| | (22) | 17.1 |
| | (23) | 17.2 |
| | (24) | 17.1 |
| | (25) | 16.9 |
| | (26) | 17.0 |
| | (27) | 16.9 |
| | (28) | 17.1 |
| | (29) | 16.7 |
| | (30) | 17.2 |
| | (31) | 17.1 |
| | (32) | 17.2 |
| | (33) | 17.2 |
| | (34) | 17.4 |
| | (35) | 17.3 |
| | (36) | 17.2 |
| | (37) | 17.2 |
| | (38) | 17.5 |
| | (39) | 17.2 |
| | (40) | 17.1 |
| | (41) | 17.3 |
| | (42) | 17.3 |
| | (43) | 17.2 |
| | (44) | 17.4 |
| | (45) | 17.3 |
| | (46) | 17.2 |
| | (47) | 17.2 |
| | (48) | 17.3 |
| | (49) | 17.3 |
| | (50) | 17.3 |
| | (51) | 17.3 |
| | (52) | 17.3 |
| | (53) | 17.3 |
| | (54) | 17.3 |
| | (55) | 17.4 |
| | (56) | 17.3 |
| | (57) | 16.9 |
| 2 weeks 40° C. | (1) | 28.0 |
| | (2) | 24.8 |
| | (3) | 25.4 |
| | (4) | 19.4 |
| | (5) | 26.7 |
| | (6) | 25.8 |
| | (7) | 23.2 |
| | (8) | 19.4 |
| | (9) | 25.8 |
| | (10) | 29.4 |
| | (11) | 26.9 |
| | (12) | 25.6 |
| | (13) | 24.3 |
| | (14) | 24.9 |
| | (15) | 27.7 |
| | (16) | 26.6 |
| | (17) | 19.9 |
| | (18) | 27.2 |
| | (19) | 19.4 |
| | (20) | 19.7 |
| | (21) | 23.5 |
| | (22) | 24.8 |
| | (23) | 25.6 |
| | (24) | 29.6 |
| | (25) | 24.3 |
| | (26) | 27.2 |
| | (27) | 23.4 |
| | (28) | 24.1 |
| | (29) | 25.3 |
| | (30) | 25.3 |
| | (31) | 24.4 |
| | (32) | 23.0 |
| | (33) | 21.1 |
| | (34) | 23.0 |
| | (35) | 22.0 |
| | (36) | 20.0 |
| | (37) | 20.8 |
| | (38) | 20.4 |
| | (39) | 20.4 |
| | (40) | 20.7 |
| | (41) | 20.5 |
| | (42) | 20.6 |
| | (43) | 20.8 |
| | (44) | 23.4 |
| | (45) | 20.2 |
| | (46) | 20.7 |
| | (47) | 20.4 |
| | (48) | 20.2 |
| | (49) | 23.5 |
| | (50) | 20.7 |
| | (51) | 20.9 |
| | (52) | 21.3 |
| | (53) | 20.7 |
| | (54) | 20.3 |
| | (55) | 23.4 |
| | (56) | 20.6 |
| | (57) | 20.3 |

After storage for two weeks at 4000, the levels of acidic species increased to 19% to 30% compared to t0 (17%) for the formulations tested within the design of experiment setup. For formulations (1) to (32) the effect of the buffer can be summarized as follows: histidine has a strong stabilizing effect and for citrate a slight destabilizing effect was observed at elevated temperatures, which decreased with decreasing pH. Hence, histidine as well as citrate (especially at lower pH) are optimal buffering systems for pembrolizumab.

For formulations (33) to (56) citrate buffer was used as a core component.

Comparing formulations (33) through (56), which contained amino acids and/or glycerol as stabilizing excipients, optimal conditions for formulations of pembrolizumab were identified in samples (36), (38), (39), (45), (47), (48) and (54). Further evaluation revealed that with a combination of high concentrations of arginine and/or lysine and 100 mM glycerol in citrate-based formulations comparable results to the sugar and/or sugar alcohol used in histidine-based formulations (1) to (32) could be achieved in terms of acidic species levels. The formulation containing Poloxamer 188 (56) yielded results comparable to polysorbate 20-containing formulations as well as the control formulation (57), which contained polysorbate 80.

After storage for one week at 40° C., after multiple freeze/thaw cycles as well as after applying mechanical stressing by orbital shaking and overhead rotation the same trends as described above were observed for formulations (1) to (57).

In addition, for the basic species the same trends were observed as for the acidic species.

### 5. Summary of Study Results

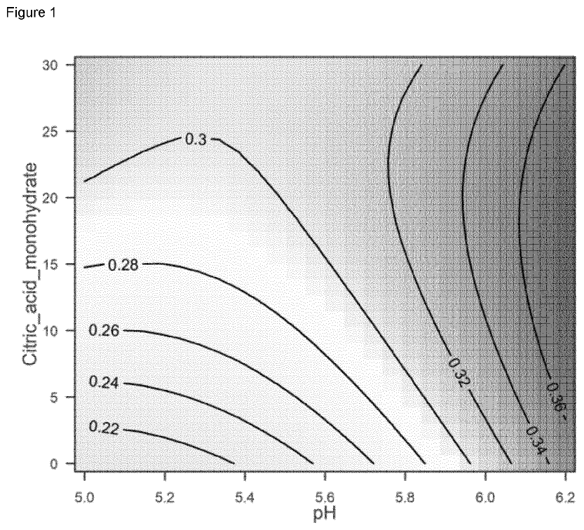

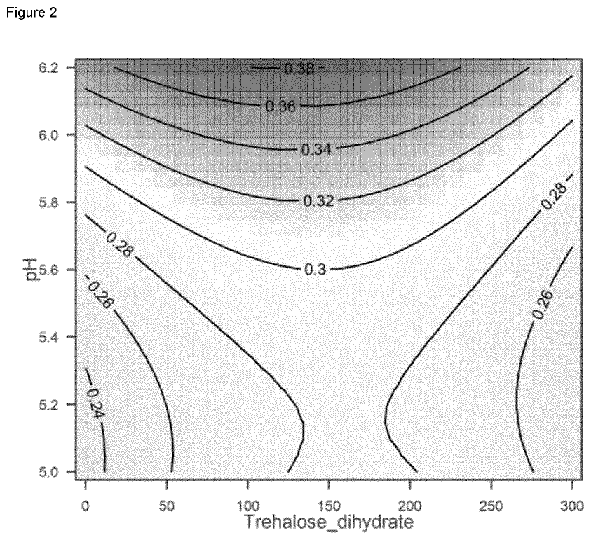

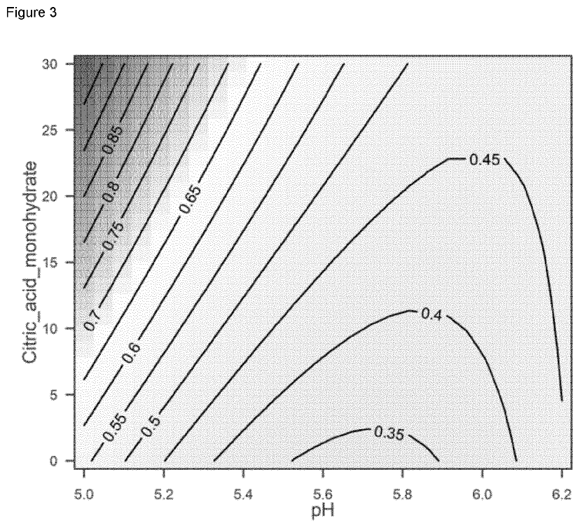

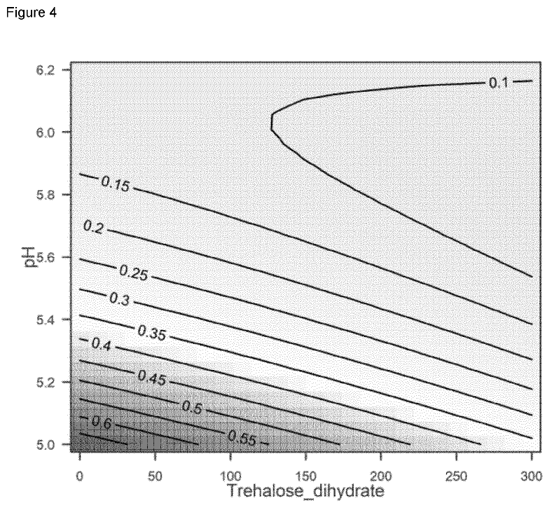

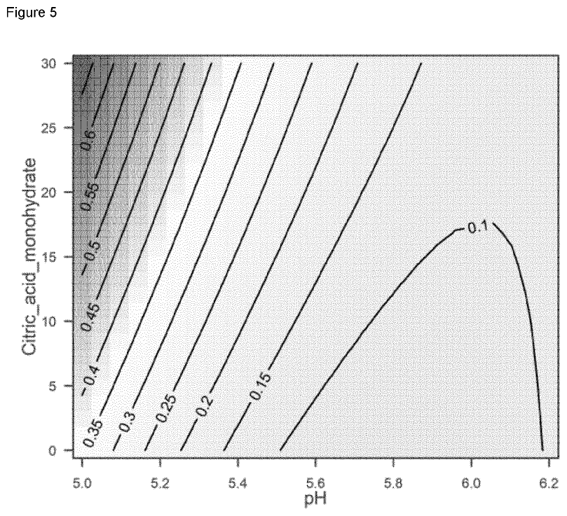

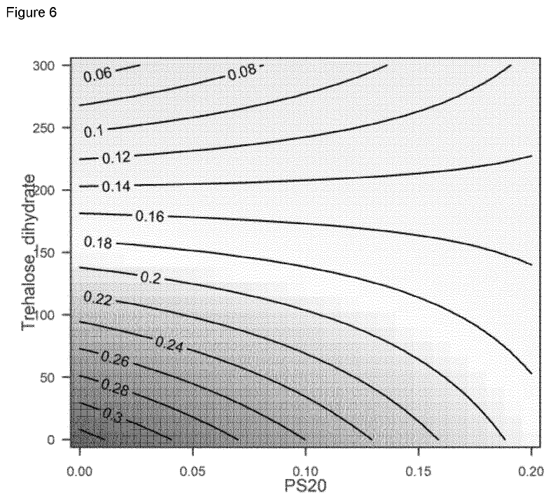

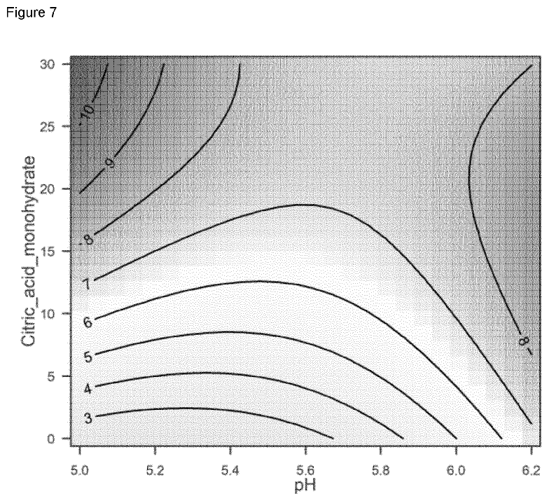

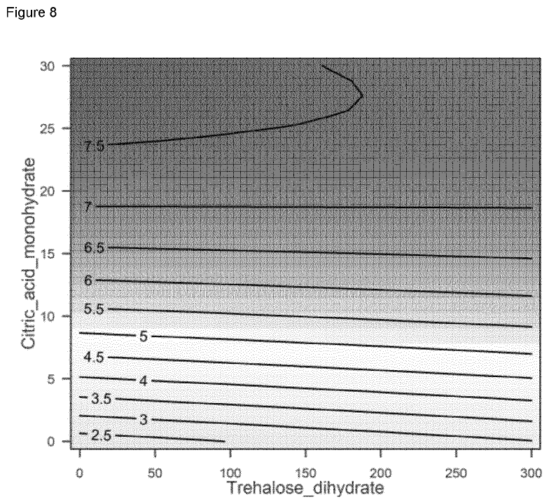

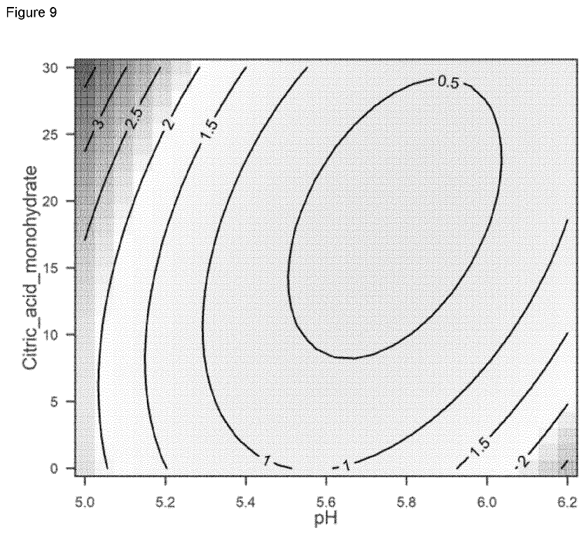

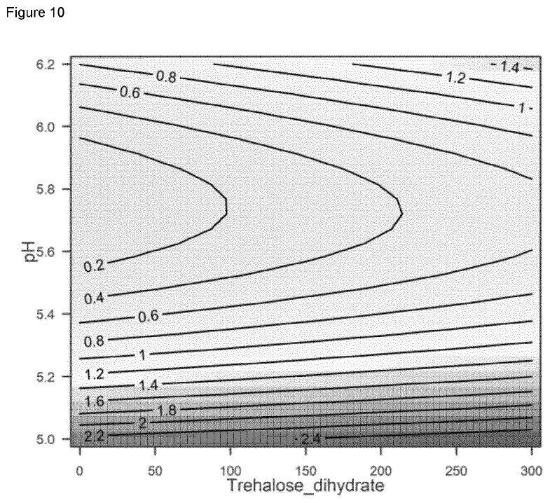

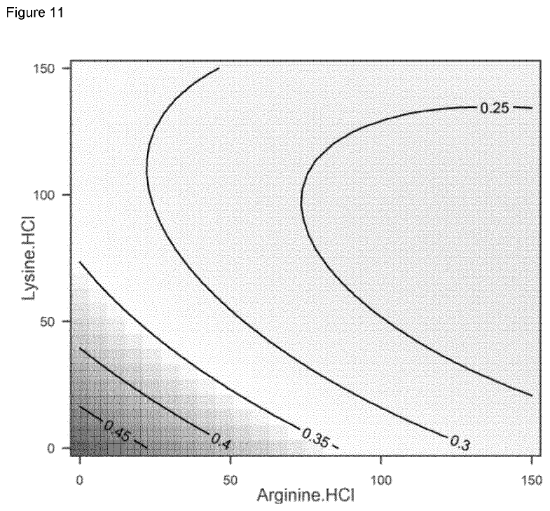

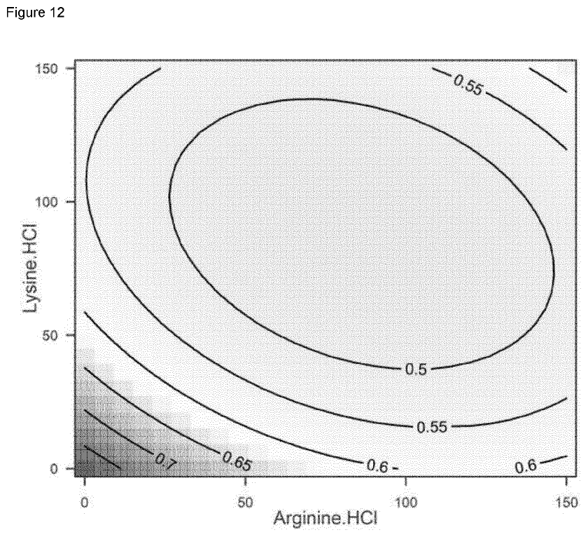

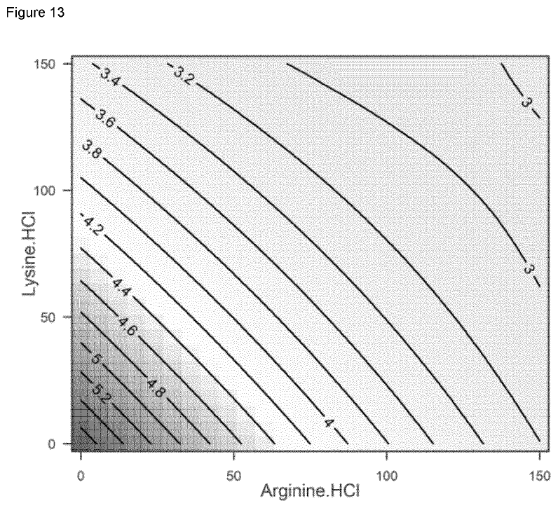

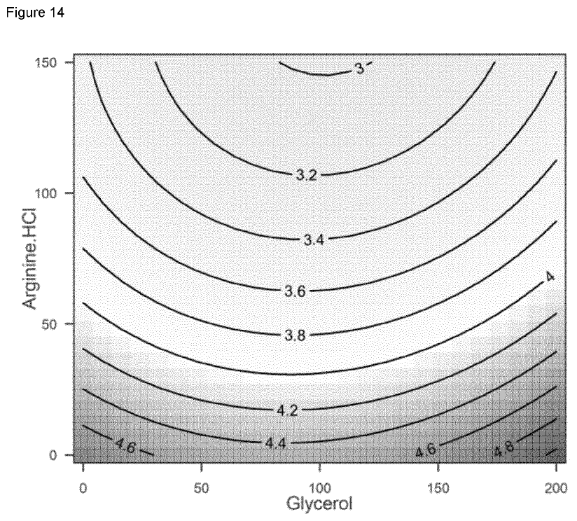

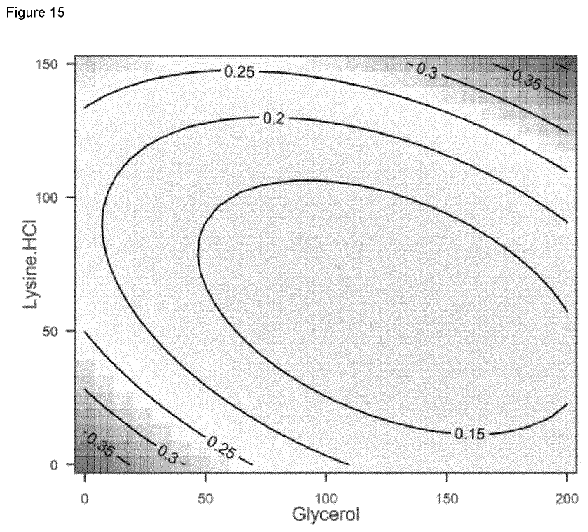

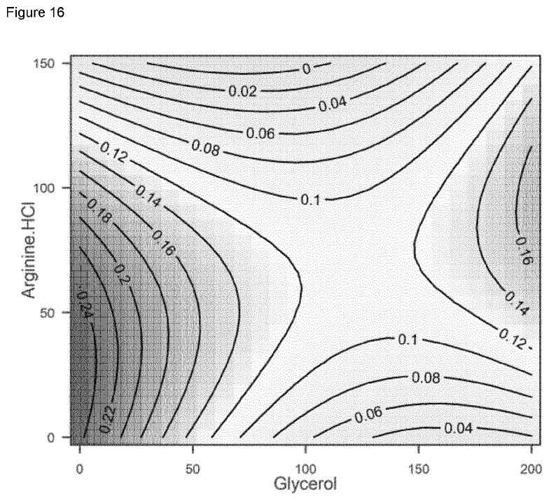

Based on the results generated with the shown method set some excipients were identified, which stabilize pembrolizumab in liquid formulations and are superior compared to other excipients tested or even compared to the reference product formulation. Formulation conditions promising stabilizing effects and the influence of the individual factors are shown in FIGS. 1 to 16.

Trehalose was found to stabilize pembrolizumab in a very broad concentration range against aggregation at elevated temperature (40° C.) as well as after applying multiple freeze/thaw cycles, whereas formulations with mannitol showed an increase in aggregation levels after freeze/thaw stressing.

In addition, a combination of high concentrations (100-300 mM) of arginine and/or lysine and 100 mM glycerol has a promising stabilizing effect on pembrolizumab against aggregation and chemical modifications like modifications leading to charge heterogeneities. Hence, a formulation of pembrolizumab without any sugar or sugar alcohol appears to be feasible. The other two amino acids tested (glycine and proline) yielded ambiguous and only small effects on the stability of pembrolizumab. Therefore, the development of further formulations tested will be centered around arginine, lysine and/or glycerol.

Histidine as well as citrate at low molarities (5-10 mM) stabilize pembrolizumab significantly. The preferred pH for histidine and citrate buffer was identified with pH 5.5. Citrate-containing formulations were also stable at lower pH values down to pH 5.0.

By using Poloxamer 188 as a surfactant, pembrolizumab formulations could be stabilized to the same extent as by using polysorbate 20 and polysorbate 80 (control formulation), respectively.

### Example 2: Formulations Selected for Stability Study

Based on the results shown in example 1, 10 formulations were selected to be tested in a short-term stability study. The same starting material as described in example 1 was used and formulations were prepared via dialysis. In addition, pembrolizumab in the Keytruda® labelled formulation buffer was included in the study as control sample (11) and dialyzed head-to-head with the other formulations. The 10 different formulations (plus control sample) are shown in Table 5.

TABLE 5
Detailed information of formulations prepared within this study (example 2)

| No. | Pembrolizumab | Buffer system | Stabilizer | Surfactant | pH |
|---|---|---|---|---|---|
| (1) | 25 mg/mL | 10 mM citric acid/ | 205 mM trehalose | 0.2 mg/mL | 5.5 |
| | | sodium citrate | | polysorbate 80 | |
| (2) | 25 mg/mL | 5 mM citric acid/ | 205 mM trehalose | 0.2 mg/mL | 5.5 |
| | | sodium citrate | | polysorbate 80 | |
| (3) | 25 mg/mL | 10 mM citric acid/ | 300 mM arginine/ | 0.2 mg/mL | 5.5 |
| | | sodium citrate | arginine HCl | polysorbate 80 | |
| (4) | 25 mg/mL | 10 mM citric acid/ | 250 mM lysine/ | 0.2 mg/mL | 5.5 |
| | | sodium citrate | lysine HCl | polysorbate 80 | |
| (5) | 25 mg/mL | 10 mM citric acid/ | 150 mM arginine/ | 0.2 mg/mL | 5.5 |
| | | sodium citrate | arginine HCl, | polysorbate 80 | |
| | | | 150 mM lysine/ | | |
| | | | lysine HCl | | |
| (6) | 25 mg/mL | 10 mM citric acid/ | 250 mM arginine/ | 0.2 mg/mL | 5.5 |
| | | sodium citrate | arginine HCl, | polysorbate 80 | |
| | | | 100 mM glycerol | | |
| (7) | 25 mg/mL | 10 mM citric acid/ | 250 mM lysine/ | 0.2 mg/mL | 5.5 |
| | | sodium citrate | lysine HCl, | polysorbate 80 | |
| | | | 100 mM glycerol | | |
| (8) | 25 mg/mL | 10 mM citric acid/ | 150 mM arginine/ | 0.2 mg/mL | 5.5 |
| | | sodium citrate | arginine HCl, | polysorbate 80 | |
| | | | 150 mM lysine/ | | |
| | | | lysine HCl, | | |
| | | | 100 mM glycerol | | |
| (9) | 25 mg/mL | 10 mM histidine/ | 300 mM arginine/ | 0.2 mg/mL | 5.5 |
| | | histidine HCl | arginine HCl | polysorbate 80 | |
| (10) | 25 mg/mL | 5 mM citric acid/ | 300 mM arginine/ | 0.2 mg/mL | 5.5 |
| | | sodium citrate | arginine HCl | polysorbate 80 | |
| (11) | 25 mg/mL | 10 mM histidine/ | 205 mM sucrose | 0.2 mg/mL | 5.5 |
| | | histidine HCl | | polysorbate 80 | |

Samples will be stored for up to 24 months under the conditions shown in Table 6. In addition, samples will undergo two conditions of mechanical stressing as well as five freeze/thaw cycles.

TABLE 6
Storage stability study of Pembrolizumab

| Stress Type/Time | | | | | | | | | | |
|---|---|---|---|---|---|---|---|---|---|---|
| Point (months) | t0 | 0.5 | 1 | 2 | 3 | 6 | 9 | 12 | 18 | 24 |
| 5° C. | X | | X | | X | X | X | X | X | X |
| 25° C. ± 2° C./ | | | X | X | X | | | | | |
| 60% RH ± 5% RH | | | | | | | | | | |
| 40° C. ± 2° C./ | | X | X | | | | | | | |
| 75% RH ± 5% RH | | | | | | | | | | |
| Mechanical stressing | | 2 conditions (orbital shaking and overhead rotation) | | | | | | | | |
| (up to 24 h) | | | | | | | | | | |
| Freeze/thaw | | 5 cycles | | | | | | | | |
| (−80° C./+25° C.) | | | | | | | | | | |

Protein stability will be determined by size exclusion chromatography (SE-HPLC) for the presence of high molecular weight species (HMWS) and by SIDS-cGE (non-reducing) for the presence of low molecular weight species (LMWS) and HMWS. Chemical modifications like glycation, oxidation and deamidation will be quantified by LC-ESI-MS and MS/MS in reduced peptide mapping. Ion exchange chromatography (IEX-HPLC) as well as imaged capillary isoelectric focusing (icIEF) will be used to detect modifications leading to charge heterogeneities. In addition, samples will be analyzed for appearance, turbidity, sub-visible particle content and particle size. The protein concentration of the samples will be determined by UV-VIS spectroscopy. The pH of the formulations will be measured at t0 only.

### Example 3: Formulations Tested in Stability Study

### 1. Sample Preparation

Based on the results obtained in Example 1, the 6 best performing formulations were selected and tested.

Pembrolizumab at 25 mg/mL was used as starting material and 7 formulations listed in Table 5 (6 alternative formulations (1)-(6) and the reference formulation (7)) were prepared via a 3-step-dialysis. Pembrolizumab in the Keytruda® labelled formulation buffer was included in the study as control sample (7) and dialyzed head-to-head with the other formulations shown in Table 5. Dialyzed pembrolizumab was adjusted to 25 mg/mL±20% and 0.22 μm sterile filtered using a PES membrane. The formulations shown to Table 5 were initially analyzed for protein concentration by UV-VIS spectroscopy at 280 nm and correct pH adjustment before starting storage stability.

TABLE 7
Detailed information of formulations prepared within this study

| No. | Pembrolizumab | Buffer system | Stabilizer | Surfactant | pH |
|---|---|---|---|---|---|
| (1) | 25 mg/mL | 10 mM citric acid/ | 300 mM arginine/ | 0.2 mg/ml | 5.5 |
| | | sodium citrate | arginine HCl | polysorbate 80 | |
| (2) | 25 mg/mL | 5 mM citric acid/ | 300 mM arginine/ | 0.2 mg/mL | 5.5 |
| | | sodium citrate | arginine HCl | polysorbate 80 | |
| (3) | 25 mg/mL | 5 mM citric acid/ | 205 mM trehalose | 0.2 mg/ml | 5.5 |
| | | sodium citrate | dihydrate | polysorbate 80 | |
| (4) | 25 mg/mL | 10 mM citric acid/ | 250 mM lysine/ | 0.2 mg/mL | 5.5 |
| | | sodium citrate | lysine HCl | polysorbate 80 | |
| (5) | 25 mg/mL | 10 mM citric acid/ | 205 mM trehalose | 0.2 mg/mL | 5.5 |
| | | sodium citrate | dihydrate | polysorbate 80 | |
| (6) | 25 mg/mL | 10 mM citric acid/ | 150 mM arginine/ | 0.2 mg/mL | 5.5 |
| | | sodium citrate | arginine HCl, | polysorbate 80 | |
| | | | 150 mM lysine/ | | |
| | | | lysine HCl | | |
| (7) | 25 mg/mL | 10 mM histidine/ | 205 mM sucrose | 0.2 mg/mL | 5.5 |
| | | histidine HCl | | polysorbate 80 | |

Storage conditions are shown in Table 8. In brief, samples were placed on storage for up to 3 months at 5° C. and 25° C./60% relative humidity (RH) as well as for up to 1 month at 40° C./75% RH. In addition, samples underwent two conditions of mechanical stressing (overhead rotation, orbital shaking) as well as five freeze/thaw cycles (−80° C./+25° C.). Sample storage will be continued for up to 24 months as described in Table 8.

TABLE 8
Storage conditions/stability program of Pembrolizumab

| | Stress Type/Time Point (months) | | | | | | | | | |
|---|---|---|---|---|---|---|---|---|---|---|
| | t0 | 0.5 | 1 | 2 | 3 | 6 | 9 | 12 | 18 | 24 |
| 5° C. | x | | x | | x | x | x | x | x | x |
| 25° C./60% | | | x | x | x | | | | | |
| RH | | | | | | | | | | |
| 40° C./75% | | x | x | | | | | | | |
| RH | | | | | | | | | | |
| Mechanical | | 2 conditions (orbital shaking at 600 rpm and overhead rotation at 30 rpm) | | | | | | | | |
| stressing | | | | | | | | | | |
| (up to 24 h) | | | | | | | | | | |
| Freeze/ | | 5 cycles | | | | | | | | |
| thaw | | | | | | | | | | |
| (−80° C./+25° C.) | | | | | | | | | | |

Protein stability was determined by size exclusion chromatography (SE-HPLC) for the presence of high molecular weight species (HMWS) and by SDS-cGE (non-reducing) for the presence of low molecular weight species (LMWS) and HMWS. Chemical modifications like glycation, oxidation and deamidation were quantified by LC-ESI-MS and -MS/MS in reduced peptide mapping. Ion exchange chromatography (IEX-HPLC) as well as imaged capillary isoelectric focusing (icIEF) were used to detect modifications leading to charge heterogeneities. In addition, samples were analyzed for appearance, turbidity, sub-visible particle content and particle size. The protein concentration of the samples was determined by UV-VIS spectroscopy. The pH of the formulations was measured at t0, after 1 month, 3 months, 6 months, 9 months and 12 months.

### 2. Analysis of Protein Content by UV-VIS, Determination of PH, Visible Particles, Subvisible Particles and Osmolality

All prepared formulations according to Table 7 were analyzed for their protein content as well as for pH and osmolality before starting the stability study (t0). All samples met the acceptance criteria for formulations according to Table 7 in protein content (25 mg/mL±10% pembrolizumab) and pH (target±0.2). No visible particles or changes in color were detected in all samples.

The pH was analyzed by the use of a SevenExcellence Multiparameter system and an InLab Micro Pro-ISM pH electrode, both from Mettler Toledo. Measurements were conducted at 23° C.-25° C. in accordance with USP using 150 μl of solution, filled in a 0.5 mL vial. For protein content determination a NanoPhotometer N120 from Implen was used. Here 2 μl of sample solution were diluted with factor 1:10 in their associated placebo buffer solution, quantified at 280 nm, using an extinction coefficient of 1.418 l/g*cm. Background correction was performed using a wavelength of 320 nm.

During the stability program all samples were not altered in their solution pH, all samples met the criteria of 5.5±0.2 in all storage conditions, also when using citrate buffer at the lower level of 5 mM. Hence, the reduced citrate concentration was sufficient for pH stability over 9 months. The pH was determined as single measurement.

Also, the concentration of pembrolizumab was stable during the complete stability program and all results were quantified within the acceptance criteria 25 mg/mL±10% pembrolizumab. The pembrolizumab concentration was determined in duplicates.

When analyzing the different formulations for their visual appearance, they all showed identical results during the stability study when comparing the formulations to each other for one storage condition. At t0 all samples were analyzed as clear and transparent. After applying a physical stress like freeze/thaw and shaking/rotation all formulations were analyzed as clear with a very slight opalescence. After 9 months incubation at 5° C. and 3 months incubation at 25° C., respectively, all formulations were inspected as clear with a slight opalescence fulfilling specifications for pembrolizumab. For visual inspection samples were analyzed in duplicates.

Subvisible particles were analyzed using a FlowCam 8100 Multi Objective from Anasysta. These micron-sized protein aggregates and particles are important quality attributes of therapeutic protein formulations due to their risk of enhancing an immunogenic response. The method was adjusted to the following parameters: efficiency of analysis: 60-70%; auto image range: 30 fps; distance to nearest neighbor: 3 μm; flow rate: 0.150 mL/min; processed sample volume: 0.100 mL.

After mechanical stress, freeze/thaw stress as well after 9 or 12 months months incubation at 5° C., 3 months at 25° C., 1 month at 40° C. all formulations perform similarly well and well within USP<788> Pharmacopoeia limits, with lowest numbers of particles in the size range 10 μm after 12 months storage at 5° C. in formulations (1) and (2), which are using arginine as stabilizer, followed by lysine containing formulation (4) and sugar containing formulations (2) and (7) with slightly increased numbers. Only a very slight increase of all particles with a size below 25 μm was observed after 1 month storage at 40° C. The results of other storage conditions remained constant within method variability. For analyzing subvisible particles by FlowCam all measurements were performed in duplicates.

### 3. Analysis of High Molecular Weight Species (HMWS) by SE-HPLC

The study samples were diluted in 20 mM histidine pH 5.5 to a final concentration of 1 mg/mL and 5 μL thereof were injected to a TSKgel UP-SW3000, (Tosoh, 4.6×150 mm, 2 μm) column to detect high molecular weight species (HMWS) of pembrolizumab.

The protein was eluted by isocratic elution using 0.1 M sodium phosphate buffer with 0.05% (v/v) sodium azide (pH 6.7) at a flow rate of 0.35 mL/min at 25° C. Eluted species were detected at a wavelength of 280 nm and displayed on a graph showing the concentration of the eluted species versus time. The elution profile showed a monomer peak with the non-aggregated protein and peaks of the protein representing higher molecular weight species (HMWS) of the protein. The areas of all peaks were determined.

Table 9 summarizes the percentage of peak area for the HMWS in relation to the total peak area of the eluted species for the formulations shown in Table 7. With start of obtaining samples after 6 months, the formulation set was reduced to the most promising candidates, the incubation of all remaining formulations was stopped at this time point. Each sample was measured in triplicates.

TABLE 9
Overview of HMWS determined using SE-HPLC

| Condition | Formulation | HMWS [%] |
|---|---|---|
| t0 | (1) | 0.8 |
| | (2) | 0.8 |
| | (3) | 0.9 |
| | (4) | 0.8 |
| | (5) | 0.9 |
| | (6) | 0.8 |
| | (7) | 0.9 |
| 24 h rotation | (1) | 0.7 |
| overhead | (2) | 0.7 |
| | (3) | 0.8 |
| | (4) | 0.7 |
| | (5) | 0.8 |
| | (6) | 0.8 |
| | (7) | 0.8 |
| 24 h orbital | (1) | 0.8 |
| shaking | (2) | 0.7 |
| | (3) | 0.9 |
| | (4) | 0.8 |
| | (5) | 0.9 |
| | (6) | 0.8 |
| | (7) | 0.8 |
| F/T | (1) | 0.7 |
| | (2) | 0.8 |
| | (3) | 0.8 |
| | (4) | 0.8 |
| | (5) | 0.8 |
| | (6) | 0.7 |
| | (7) | 0.7 |
| 2 weeks 40° C. | (1) | 0.9 |
| | (2) | 0.9 |
| | (3) | 1.2 |
| | (4) | 1.0 |
| | (5) | 1.2 |
| | (6) | 0.9 |
| | (7) | 1.0 |
| 1 month 5° C. | (1) | 0.7 |
| | (2) | 0.7 |
| | (3) | 1.0 |
| | (4) | 0.7 |
| | (5) | 0.9 |
| | (6) | 0.7 |
| | (7) | 0.8 |
| 1 month 25° C. | (1) | 0.8 |
| | (2) | 0.8 |
| | (3) | 1.1 |
| | (4) | 0.8 |
| | (5) | 1.0 |
| | (6) | 0.8 |
| | (7) | 0.8 |
| 1 month 40° C. | (1) | 1.0 |
| | (2) | 1.0 |
| | (3) | 1.4 |
| | (4) | 1.2 |
| | (5) | 1.4 |
| | (6) | 1.1 |
| | (7) | 1.3 |
| 2 months 25° C. | (1) | 0.9 |
| | (2) | 0.9 |
| | (3) | 1.1 |
| | (4) | 0.9 |
| | (5) | 1.1 |
| | (6) | 0.9 |
| | (7) | 1.0 |
| 3 months 5° C. | (1) | 0.7 |
| | (2) | 0.8 |
| | (3) | 0.9 |
| | (4) | 0.8 |
| | (5) | 0.9 |
| | (6) | 0.8 |
| | (7) | 0.9 |
| 3 months 25° C. | (1) | 0.9 |
| | (2) | 0.9 |
| | (3) | 1.2 |
| | (4) | 1.0 |
| | (5) | 1.1 |
| | (6) | 0.9 |
| | (7) | 1.0 |
| 6 months 5° C. | (1) | 0.7 |
| | (2) | 0.7 |
| | (3) | 1.0 |
| | (4) | 0.8 |
| | (5) | n.a.¹⁾ |
| | (6) | n.a.¹⁾ |
| | (7) | 1.0 |
| 9 months 5° C. | (1) | 0.8 |
| | (2) | 0.8 |
| | (3) | 1.1 |
| | (4) | 0.9 |
| | (5) | n.a.¹⁾ |
| | (6) | n.a.¹⁾ |
| | (7) | 1.0 |
| 12 months 5° C. | (1) | 0.8 |
| | (2) | 0.8 |
| | (3) | 1.0 |
| | (4) | 0.8 |
| | (5) | n.a.¹⁾ |
| | (6) | n.a.¹⁾ |
| | (7) | 1.0 |

¹⁾Stability study stopped for this formulation after 3 months

After storage for up to 9 months at 5° C. (target storage condition), the HMWS remained at t0 level for all formulations tested within this stability study promising long term storage stability at this condition. Slightly better results were achieved by using formulations (1) and (2) containing arginine as stabilizer. After storage for up to 12 months at 5° C. (target storage condition) slightly better results were achieved by using formulations (1) and (2) containing arginine as stabilizer, and (4) containing lysine. After storage for up to 3 months at 25° C., the HMWS levels slightly increased to 0.9% to 1.2% compared to t0 (0.8 and 0.9%, respectively). Lowest HMWS levels after storage for up to 3 months at 25° C. were observed for formulations (1), (2) and (6), which contain the amino acid arginine (either arginine only or in combination with lysine), followed by formulation (4), which contains lysine only. The trehalose containing formulations (3) and (5) showed a slight increase in HMWS levels after storage for up to 3 months at 25° C., but at the target storage condition surprisingly no relevant increase of HMWS was detected. In addition, all formulations tested remained stable after multiple freeze/thaw (F/T) cycles and after applying mechanical stress. Overall, all formulations tested within this study showed similar or improved results compared to the reference formulation (7).

### 4. Detection of Acidic and Basic Species by IEX-HPLC

The study samples were diluted in eluent A (20 mM MES, pH 6.2) to a final concentration of 1 mg/mL and 30 μL thereof were injected onto a MabPac SCX-10 (Thermo Scientific, 4×250 mm, 10 μm) column in order to detect modifications of pembrolizumab leading to charge heterogeneities. Proteins were eluted using a mixture of mobile phase A (20 mM MES, pH 6.2) and B (20 mM MES, 120 mM KCl, pH 6.2) at a flow rate of 1 mL/min at 40° C. by applying a salt gradient (KCl). Eluted species were detected at a wavelength of 280 nm and displayed on a graph showing the concentration of the eluted species versus time. The elution profile showed a main peak as well as several peaks representing the acidic and basic variants. The areas of all peaks were determined. Table 10 summarizes the percentage of the peak area for the acidic species in relation to the total peak area of the eluted species for the formulations shown in Table 7. Each sample was measured in triplicates.

TABLE 10
Overview of acidic species determined using IEX-HPLC

| | | Acidic species | Basic species |
|---|---|---|---|
| Condition | Formulation | [%] | [%] |
| t0 | (1) | 17.4 | 30.4 |
| | (2) | 17.4 | 30.4 |
| | (3) | 17.6 | 30.4 |
| | (4) | 17.4 | 30.5 |
| | (5) | 17.5 | 30.5 |
| | (6) | 17.4 | 30.6 |
| | (7) | 17.8 | 30.4 |
| 24 h rotation | (1) | 17.4 | 30.4 |
| overhead | (2) | 17.3 | 30.7 |
| | (3) | 17.6 | 30.3 |
| | (4) | 17.4 | 30.5 |
| | (5) | 17.5 | 30.5 |
| | (6) | 17.2 | 30.7 |
| | (7) | 17.4 | 30.8 |
| 24 h orbital | (1) | 17.2 | 30.5 |
| shaking | (2) | 17.2 | 30.7 |
| | (3) | 17.5 | 30.4 |
| | (4) | 17.2 | 30.6 |
| | (5) | 17.4 | 30.4 |
| | (6) | 17.2 | 30.6 |
| | (7) | 17.3 | 30.9 |
| F/T | (1) | 17.5 | 30.2 |
| | (2) | 17.4 | 30.4 |
| | (3) | 17.6 | 30.1 |
| | (4) | 17.4 | 30.5 |
| | (5) | 17.6 | 30.2 |
| | (6) | 17.4 | 30.4 |
| | (7) | 17.6 | 30.4 |
| 2 weeks 40° C. | (1) | 22.2 | 28.3 |
| | (2) | 21.8 | 28.9 |
| | (3) | 25.7 | 25.3 |
| | (4) | 22.5 | 28.5 |
| | (5) | 26.7 | 24.2 |
| | (6) | 22.2 | 28.5 |
| | (7) | 23.3 | 28.7 |
| 1 month 5° C. | (1) | 17.2 | 30.1 |
| | (2) | 17.2 | 30.1 |
| | (3) | 17.6 | 29.9 |
| | (4) | 17.2 | 30.2 |
| | (5) | 17.5 | 29.9 |
| | (6) | 17.1 | 30.2 |
| | (7) | 17.8 | 29.8 |
| 1 month 25° C. | (1) | 17.5 | 29.8 |
| | (2) | 17.5 | 30.0 |
| | (3) | 18.7 | 28.1 |
| | (4) | 17.6 | 29.7 |
| | (5) | 19.0 | 27.6 |
| | (6) | 17.6 | 29.8 |
| | (7) | 18.3 | 29.1 |
| 1 month 40° C. | (1) | 30.2 | 25.1 |
| | (2) | 29.9 | 26.2 |
| | (3) | 36.3 | 20.9 |
| | (4) | 30.7 | 25.4 |
| | (5) | 37.6 | 20.0 |
| | (6) | 30.3 | 25.5 |
| | (7) | 33.2 | 25.8 |
| 2 months 25° C. | (1) | 18.7 | 28.9 |
| | (2) | 18.6 | 29.3 |
| | (3) | 20.2 | 26.1 |
| | (4) | 18.8 | 29.0 |
| | (5) | 20.9 | 25.4 |
| | (6) | 18.7 | 29.0 |
| | (7) | 19.5 | 28.0 |
| 3 months 5° C. | (1) | 17.1 | 30.4 |
| | (2) | 17.1 | 30.3 |
| | (3) | 17.5 | 30.0 |
| | (4) | 17.1 | 30.5 |
| | (5) | 17.6 | 29.9 |
| | (6) | 17.1 | 30.4 |
| | (7) | 18.0 | 29.9 |
| 3 months 25° C. | (1) | 20.1 | 28.7 |
| | (2) | 20.3 | 29.0 |
| | (3) | 23.2 | 24.7 |
| | (4) | 20.4 | 28.5 |
| | (5) | 23.9 | 23.8 |
| | (6) | 20.3 | 28.5 |
| | (7) | 22.3 | 27.3 |
| 6 months 5° C. | (1) | 17.0 | 30.3 |
| | (2) | 16.9 | 30.6 |
| | (3) | 17.6 | 29.6 |
| | (4) | 17.0 | 30.2 |
| | (5) | n.a.¹⁾ | n.a.¹⁾ |
| | (6) | n.a.¹⁾ | n.a.¹⁾ |
| | (7) | 17.4 | 29.8 |
| 9 months 5° C. | (1) | 17.0 | 30.5 |
| | (2) | 16.9 | 30.8 |
| | (3) | 17.6 | 29.3 |
| | (4) | 17.1 | 30.3 |
| | (5) | n.a.¹⁾ | n.a.¹⁾ |
| | (6) | n.a.¹⁾ | n.a.¹⁾ |
| | (7) | 17.4 | 29.6 |
| 12 months 5° C. | (1) | 17.1 | 29.7 |
| | (2) | 16.9 | 30.1 |
| | (3) | 17.8 | 28.2 |
| | (4) | 17.1 | 29.5 |
| | (5) | n.a.¹⁾ | n.a.¹⁾ |
| | (6) | n.a.¹⁾ | n.a.¹⁾ |
| | (7) | 17.5 | 29.2 |

¹⁾Stability study stopped for this formulation after 3 months

After storage for up to 3 months at 5° C. (target storage condition), both the acidic species and basic species remained at t0 level for all formulations tested within this stability study promising long term stability. After storage for up to 9 months at 5° C. (target storage condition), both the acidic species and basic species increased only slightly. After storage for up to 12 months at 5° C. (target storage condition), both the acidic species and basic species remained at t0 level for all formulations tested within this stability study promising long term stability, lowest variations compared to t0 were shown in formulations (1), (2) and (4) which contain one or two amino acids (either arginine or lysine or both arginine and lysine). After storage for up to 3 months at 25° C., the acidic species levels slightly increased to 20.1% to 23.9% compared to t0 (about 17.5%). Lowest acidic species levels after storage were observed for formulations (1), (2), (4) and (6), which contain one or two amino acids (either arginine or lysine or both arginine and lysine). The trehalose containing formulations (3) and (5) showed only a slight increase in acidic species levels, which is comparable to the reference formulation (7). In addition, all formulations tested remained stable after multiple freeze/thaw cycles and after applying mechanical stress.

For the generation of basic species an identical trend was observed. After storage for up to 3 months at 25° C., the basic species levels slightly decreased to 23.8% to 29.0% compared to t0 (30.4%-30.6%). Best results (here: highest contents) of basic species levels after storage were again observed for formulations (1), (2), (4) and (6), which contain one or two amino acids (either arginine or lysine or both arginine and lysine). The trehalose containing formulations (3) and (5) showed only a slight decrease in basic species levels, which is comparable to the reference formulation (7). Overall, all formulations tested within this study showed similar or better results compared to the reference formulation (7).

### 5. Analysis of Low Molecular Weight Species (LMWS) by SDS-cGE (Non-Red.)

Capillary gel electrophoresis for quantification of LMWS was carried out using a LabChip GX II Touch Protein Characterization System and the Protein Express assay kit (Cat #CLS960008), both Perkin Elmer.

For preparing the sample denaturing solution 5400 μl of Protein Express sample buffer from the assay kit were mixed with 300 μl 10% LDS (Lithium Dodecylsulfate, solved in water) and 300 μl of a 200 mmol/L solution of NEM (N-Ethylmaleimide, solved in water). 7 μl of this denaturing solution were mixed with 2 μl of pembrolizumab containing samples (prediluted to 2 mg/mL with water). For denaturation the sample was incubated at 75° C. for 10 minutes. After that 35 μl of water were added to each sample immediately before analysis. Analysis was performed by using the assay presets from method “P200 Antibody Analysis” of Perkin Elmer instrument software.

For the cGE Method, separation was performed by forward injection in a neutral, bare fused silica capillary (20 cm effective length and 50 μm diameter) with a PA800 plus instrument from Beckman Coulter. After voltage forced application of the sample (5 kV for 20 s) into the capillary, protein separation was performed by applying a voltage of 15 kV for 30 min in case of reducing conditions and 15 kV for 40 min in case of non-reducing conditions. UV absorption was measured at 220 nm using the PDA detector and a 100×200 aperture. The capillary temperature was kept constant at 25° C. for all steps. The autosampler temperature was set to 15° C. Data were evaluated in terms of peak integration using the 32Karat software (Beckman Coulter). Peak areas were determined as velocity-corrected relative peak areas, considering the fact that in capillary electrophoresis early peaks migrate faster through the detector window than later peaks. Sample peak integration was performed in comparison to the electropherogram of a formulation buffer or pure water blank to identify and exclude non-protein-specific peaks.

Table 11 summarizes the percentage of the peak area for the LMWS in relation to the total peak area of the eluted species for the formulations shown in Table 7. Each sample was measured in triplicates.

TABLE 11
Overview of LMWS determined using SDS-cGE (non-red.)

| Condition | Formulation | LMWS [%] |
|---|---|---|
| t0 | (1) | 0.4 |
| | (2) | 0.5 |
| | (3) | 0.4 |
| | (4) | 0.5 |
| | (5) | 0.4 |
| | (6) | 0.4 |
| | (7) | 0.4 |
| 24 h rotation | (1) | 0.7 |
| overhead | (2) | 0.7 |
| | (3) | 0.7 |
| | (4) | 0.7 |
| | (5) | 0.6 |
| | (6) | 0.7 |
| | (7) | 0.6 |
| 24 h orbital | (1) | 0.7 |
| shaking | (2) | 0.6 |
| | (3) | 0.6 |
| | (4) | 0.6 |
| | (5) | 0.6 |
| | (6) | 0.7 |
| | (7) | 0.6 |
| F/T | (1) | 0.8 |
| | (2) | 0.7 |
| | (3) | 0.7 |
| | (4) | 0.7 |
| | (5) | 0.7 |
| | (6) | 0.8 |
| | (7) | 0.8 |
| 2 weeks 40° C. | (1) | 1.0 |
| | (2) | 1.0 |
| | (3) | 0.9 |
| | (4) | 1.1 |
| | (5) | 0.8 |
| | (6) | 1.0 |
| | (7) | 0.9 |
| 1 month 5° C. | (1) | 0.7 |
| | (2) | 0.7 |
| | (3) | 0.7 |
| | (4) | 0.7 |
| | (5) | 0.7 |
| | (6) | 0.7 |
| | (7) | 0.7 |
| 1 month 25° C. | (1) | 1.0 |
| | (2) | 0.8 |
| | (3) | 0.8 |
| | (4) | 1.0 |
| | (5) | 0.8 |
| | (6) | 0.9 |
| | (7) | 0.8 |
| 1 month 40° C. | (1) | 2.2 |
| | (2) | 2.2 |
| | (3) | 1.9 |
| | (4) | 2.3 |
| | (5) | 1.8 |
| | (6) | 2.3 |
| | (7) | 1.9 |
| 3 months 5° C. | (1) | 0.7 |
| | (2) | 0.7 |
| | (3) | 0.7 |
| | (4) | 0.8 |
| | (5) | 0.8 |
| | (6) | 0.7 |
| | (7) | 0.7 |
| 3 months 25° C. | (1) | 1.2 |
| | (2) | 1.1 |
| | (3) | 1.1 |
| | (4) | 1.1 |
| | (5) | 1.0 |
| | (6) | 1.1 |
| | (7) | 1.0 |
| 6 months 5° C. | (1) | 0.9 |
| | (2) | 0.7 |
| | (3) | 0.6 |
| | (4) | 0.8 |
| | (5) | n.a.¹⁾ |
| | (6) | n.a.¹⁾ |
| | (7) | 0.7 |
| 9 months 5° C. | (1) | 0.6 |
| | (2) | 0.6 |
| | (3) | 0.6 |
| | (4) | 0.6 |
| | (5) | n.a.¹⁾ |
| | (6) | n.a.¹⁾ |
| | (7) | 0.5 |
| 12 months 5° C. | (1) | 0.6 |
| | (2) | 0.6 |
| | (3) | 0.6 |
| | (4) | 0.7 |
| | (5) | n.a.¹⁾ |
| | (6) | n.a.¹⁾ |
| | (7) | 0.6 |

¹⁾Stability study stopped for this formulation after 3 months

After storage for up to 3 months at 5° C. (target storage condition), the LMWS just slightly increased to about 0.7% compared to t0 (about 0.4%) for all formulations tested within this stability study promising successful long term storage stability. Essentially the same observation was made after storage for 9 months at 5° C. After storage for up to 12 months at 5° C. (target storage condition), the LMWS just slightly increased to about 0.6% to 0.7% compared to t0 (about 0.4%) for all formulations tested within this stability study promising successful long term storage stability. After storage for up to 3 months at 25° C., the LMWS levels increased to about 1.1% compared to t0 (ca. 0.4%) without any significant difference between the formulations tested. In addition, all formulations remained stable after multiple freeze/thaw cycles and after applying mechanical stress. Overall, all formulations tested within this study showed similar very good results and are similar compared to the reference formulation (7).

### 6. Detection of Modifications by LC-ESI-MS and -MS/MS

Analysis by LC-ESI-MS and -MS/MS was used for sequencing pembrolizumab by peptide mapping. Several post translational modifications (oxidation, deamidation and glycation) were quantified using a combination of several different digestion conditions followed by LC-ESI-MS and -MS/MS measurement. LC-ESI-MS and -MS/MS mass spectra were obtained using the UltiMate® 3000 system (Thermo Fisher Scientific) coupled to a Q Exactive Orbitrap Plus mass spectrometer (Thermo Fisher Scientific). The separation of the peptides was performed by reversed phase (RP) chromatography on an Accucore RP-MS LC column (2.1×100 mm, 2.6 μm particle size, Thermo Fisher Scientific). The following eluents were used: A: water with 0.1% formic acid; B: acetonitrile with 0.1% formic acid. A segmented gradient from 3% B to 36% B in 45 min at 30° C. with a flow rate of 0.4 mL/min was applied. MS and MS/MS spectra (produced with Higher Energy Collisional Dissociation, HCD) were recorded in positive ion mode with internal mass calibration. The datasets were searched with Protein Metrics Byonic™ against a sequence database displaying the pembrolizumab sequence and common contaminants (e.g. sequences of proteases used for the digestion) and analyzed for the deamidation level of asparagine/glutamine, the oxidation level of methionine/tryptophan and the glycation level. Samples from the same stability pull point were proteolytically digested, reduced and alkylated at the same time.

Samples shown in Table 7 were analyzed as single measurement before and after 1-month incubation at 40° C./75% relative humidity. The pull point after a one month incubation at 40° C. was chosen for evaluation of trends at accelerated conditions, which will also occur at target storage temperature but with slower velocity. After 3 months at 5° C. modifications would probably not exceed method variations. Hence, for this storage condition the analysis of later pull points is more relevant.

Table 12 summarizes the deamidation levels, Table 13 the oxidation levels and Table 14 the glycation levels for the formulations shown in Table 1.

TABLE 12
Overview of deamidation levels determined
using LC-ESI-MS and -MS/MS

| Condition | Formulation | Total deamidation [%] |
|---|---|---|
| t0 | (1) | 4.10 |
| | (2) | 4.85 |
| | (3) | 5.05 |
| | (4) | 4.68 |
| | (5) | 4.11 |
| | (6) | 4.83 |
| | (7) | 4.81 |
| 1 month 40° C. | (1) | 24.64 |
| | (2) | 25.97 |
| | (3) | 33.29 |
| | (4) | 32.70 |
| | (5) | 36.28 |
| | (6) | 30.61 |
| | (7) | 32.13 |

TABLE 13
Overview of oxidation levels determined
using LC-ESI-MS and -MS/MS

| Condition | Formulation | Total oxidation [%] |
|---|---|---|
| t0 | (1) | 16.86 |
| | (2) | 17.80 |
| | (3) | 20.45 |
| | (4) | 17.96 |
| | (5) | 17.71 |
| | (6) | 17.35 |
| | (7) | 16.98 |
| 1 month 40° C. | (1) | 28.71 |
| | (2) | 28.53 |
| | (3) | 26.19 |
| | (4) | 37.41 |
| | (5) | 26.71 |
| | (6) | 33.18 |
| | (7) | 37.55 |

TABLE 14
Overview of glycation levels determined
using LC-ESI-MS and -MS/MS

| Condition | Formulation | Total glycation [%] |
|---|---|---|
| t0 | (1) | 1.66 |
| | (2) | 1.72 |
| | (3) | 1.68 |
| | (4) | 1.62 |
| | (5) | 2.08 |
| | (6) | 1.79 |
| | (7) | 1.71 |
| 1 month | (1) | 1.39 |
| 40° C. | (2) | 1.37 |
| | (3) | 1.57 |
| | (4) | 1.50 |
| | (5) | 1.50 |
| | (6) | 1.47 |
| | (7) | 2.60 |

Total deamidation levels strongly increased after storage for 1 month at 40° C. The lowest content of deamidation was obtained when using formulations (1) and (2) which both are based on the use of arginine. All other formulations led to higher levels of total deamidation after storage.

Total oxidation levels increased after storage for 1 month at 40° C. A slightly smaller increase was observed for trehalose containing formulations (3) and (5), and for formulations using arginine without lysine, i.e. formulations (1) and (2). Highest oxidation levels were observed for lysine containing formulations (4) and (6) and the reference formulation (7).

Glycation levels were still within method variation after storage for 1 month at 40° C. without any significant differences between the alternative formulations. In contrast, reference formulation (7) which uses sucrose as excipient showed a higher glycation level after storage compared to all alternative formulations.

### 7. Summary of Study Results

Based on the results obtained in this stability program which were analyzed with the shown method set, novel compositions could be identified which can stabilize pembrolizumab in liquid formulations and are superior compared to other excipients tested or even to the reference product formulation.

Especially the use of arginine improved stability of pembrolizumab at 25 mg/mL. Formulations (1) and (2), comprising 300 mM arginine, 0.2 mg/mL polysorbate 80, 5 mmol/L or 10 mmol/L citric acid at a pH 5.5 led to very promising results, especially with respect to chemical stability of pembrolizumab. For example, IEX-HPLC showed best stability of pembrolizumab in formulations (1) and (2) with regard to acidic and basic species, also deamidation analyzed by peptide mapping was lowest in formulations (1) and (2) when compared to all other tested formulations. Similar effects could be demonstrated when arginine was replaced by the amino acid lysine (4) or mixed with lysine (6). Also generation of HMWS was lowest when using formulations (1) and (2), both based on the use of arginine as stabilizer.

But also the use of trehalose in the formulations (3) and (5) led to promising stability data when drug product is stored at the target storage temperature of 2° C. to 8° C. Here, all relevant stability data were stable and comparable to other tested formulations including the reference formulation. Anyway, using the reference formulation led to higher unwanted glycation when compared to the other formulations, due to the presence of sucrose.

The use of the buffer system citric acid/sodium citrate showed a slight benefit when the concentration was reduced to 5 mM, in particular in combination with trehalose. Also, the reduced buffer concentration or the buffer capacity at pH 5.5, respectively, was successful in stabilizing the pH during the stability program.

### II. Formulation Development of Highly-Concentrated Pembrolizumab Formulations

### Example 1: Sample Preparation of Highly Concentrated Pembrolizumab in a Histidine Buffered Solution

Ultrafiltration/diafiltration (UF/DF) operations were employed to prepare the desired therapeutic monoclonal antibody (mAb) formulations. Here, pembrolizumab was transferred by UF/DF and subsequent spiking of polysorbate 20 into a formulation containing 10 mM L-histidine/histidine HCl, 205 mM Trehalose dihydrate, 0.01% (w/v) polysorbate 20, pH 5.5, and 100 mg/mL or 150 mg/mL pembrolizumab by the following steps:

13 grams of pembrolizumab at a concentration of 11.4 mg/mL were concentrated by UF/DF and transferred into a formulation containing 10 mM L-histidine/histidine HCl, 205 mM Trehalose dihydrate, pH 5.5 to a target concentration of 120 mg/mL. For this step a Pellicon® 3 Cassette with Biomax® 30 kDa Membrane, D screen was used (Merck Millipore), providing a membrane load of approximately 13 g pembrolizumab/264 cm². As process condition 1 bar transmembrane pressure was applied with a 6 L/m²/min feed flow rate. Buffer exchange was carried out for ≤5 diafiltration volumes with formulation buffer without polysorbate 20.

After that the solution containing now 120 mg/mL pembrolizumab was sterile filtered by using a Sartopore 2 XLG 0.22 μm PES filter (filter area 210 cm²) without pre-flush of the filter. The final concentration of pembrolizumab was quantified to be 116.9 mg/mL with good step recovery of 91.6%.

After that a 50:50 split of this solution was conducted. The first part was diluted with 10 mM L-histidine/histidine HCl, 205 mM Trehalose dihydrate, pH 5.5 to a final concentration of 100 mg/mL pembrolizumab in 10 mM L-histidine/histidine HCl, 205 mM Trehalose dihydrate, pH 5.5, and polysorbate 20 was added to provide a final concentration of 0.01% (w/v) polysorbate 20 in the solution.

The second part was transferred into a Vivaspin filter (Sartorius Stedim) using a 30 kDa PES membrane and the concentration was successfully increased to 170 mg/mL pembrolizumab. After that the concentration was adjusted to 150 mg/mL pembrolizumab by dilution with 10 mM L-histidine/histidine HCl, 205 mM Trehalose dihydrate, pH 5.5, followed by sterile filtration using a bottle top vacuum filter (PES filter membrane area: 13.6 μm) with a step recovery of 95%. Finally, polysorbate 20 was spiked into the formulation to a final concentration of 0.01% (w/v).

Table 15 shows analytical results covering the different steps during preparation of the samples and the final processed samples comprising a pembrolizumab concentration of about 100 mg/mL or 150 mg/mL. The process was very gentle and smooth, HMWS determined by SE-HPLC showed just a slight increase from 0.84% HMWS to 1.10% in the 100 mg/mL pembrolizumab sample and comparable 1.08% in the 150 mg/mL sample. Other impurities like LMWS (quantified by CE-SDS non-reduced), acidic and basic species (quantified by HP-CEX) were also in an acceptable range and not altered by the processing steps. With getting the solutions successfully sterile filtered through a 0.22 μm filter with negligible loss of material, pembrolizumab in the described histidine buffered solution is a very good formulation candidate suitable for achieving high protein concentrations in a scalable manufacturing process. All methods are described in example 11.4.

TABLE 15
Preparation of highly concentrated pembrolizumab by UF/DF and characterization of samples

| | | | | Δ HMWS | | | | | | |
|---|---|---|---|---|---|---|---|---|---|---|
| | | | | increase | | LMWS | Main Peak | Acidic | Main | |
| | | Concentration | HMWS | compared to | | (CE-SDS | (CE-SDS | species | peak | Basic |
| | | pembrolizumab | (SE- | Sample | Main Peak | non | non | (HP- | (HP | species |
| Sample ID | Description | (UV280) | HPLC) | before UF/DF | (SE-HPLC) | reduced) | reduced) | CEX) | CEX) | (HP-CEX) |
| IB037.02- | Sample before | 11.54 mg/mL | 0.84% | 0.0% | 99.2% | n.a. | n.a. | n.a. | n.a. | n.a. |
| UFDF-S | UF/DF | | | | | | | | | |
| IB037.02- | Process control 40 | 39.37 mg/mL | 0.83% | −0.1% | 99.2% | n.a. | n.a. | n.a. | n.a. | n.a. |
| DF 40 g/L | mg/mL | | | | | | | | | |
| IB037.02- | Process control 80 | 78.43 mg/mL | 0.96% | 0.12% | 99.0% | n.a. | n.a. | n.a. | n.a. | n.a. |
| DF 80 g/L | mg/mL | | | | | | | | | |
| IB037.02- | Sample after | 118.82 mg/mL | 1.15% | 0.31% | 98.9% | n.a. | n.a. | n.a. | n.a. | n.a. |
| DF 120 g/L | UF/DF | | | | | | | | | |
| IB037.02- | Sample after | 169.25 mg/mL | 1.16% | 0.32% | 98.8% | n.a. | n.a. | n.a. | n.a. | n.a. |
| UF3-E 170 | Vivaspin | | | | | | | | | |
| g/L | concentration | | | | | | | | | |
| IB037.03- | Sample at target | 149.29 mg/mL | 1.08% | 0.24% | 98.9% | 1.97% | 98.03% | 14.7% | 53.5% | 31.9% |
| DS 150 g/L | 150 mg/mL, | | | | | | | | | |
| | formulated in 10 | | | | | | | | | |
| | mM L-histidine/ | | | | | | | | | |
| | histidine HCl, 205 | | | | | | | | | |
| | mM Trehalose | | | | | | | | | |
| | dihydrate, 0.01% | | | | | | | | | |
| | polysorbate 20, | | | | | | | | | |
| | pH 5.5, 0.22 μm | | | | | | | | | |
| | filtrated, sample | | | | | | | | | |
| | (b) | | | | | | | | | |
| IB037.04- | Sample at target | 100.99 mg/mL | 1.10% | 0.26% | 98.9% | 2.38% | 79.63% | 14.9% | 52.8% | 32.3% |
| DS 100 g/L | 100 mg/mL, | | | | | | | | | |
| | formulated in 10 | | | | | | | | | |
| | mM L-histidine/ | | | | | | | | | |
| | histidine HCl, 205 | | | | | | | | | |
| | mM Trehalose | | | | | | | | | |
| | dihydrate, 0.01% | | | | | | | | | |
| | polysorbate 20, | | | | | | | | | |
| | pH 5.5, sample (a) | | | | | | | | | |

### Example 2: Freeze/Thaw Stability of Pembrolizumab in Concentrations of 100 mg/mL or 150 mg/mL in a Histidine-Buffered Solution

Samples produced in example 1 and shown in Table 16 were tested for stability against freeze/thaw stress and analyzed with regard to the presence of HMWS by SE-HPLC (method described in example 4).

TABLE 16
Detailed information of formulations used in this study

| | Pembro- | Buffer | | | |
|---|---|---|---|---|---|
| Sample | lizumab | system | Stabilizer | Surfactant | pH |
| (a) | 100 mg/mL | 10 mM | 205 mM | 0.1 mg/mL | pH 5.5 |
| | | L-histidine/ | trehalose | polysorbate | |
| | | histidine HCl | dihydrate | 20 | |
| (b) | 150 mg/mL | 10 mM | 205 mM | 0.1 mg/mL | pH 5.5 |
| | | L-histidine/ | trehalose | polysorbate | |
| | | histidine HCl | dihydrate | 20 | |

For this, 500 μl aliquots of both samples (a) and (b) were analyzed before and after a freezing process to ≤−65° C. by SE-HPLC (according to the method described in example 3).

TABLE 17
Overview of HMWS determined via SE-HPLC
before and after freeze/thaw process

| | | HMWS [%] | HMWS [%] |
|---|---|---|---|
| Sample | Pembrolizumab | Before freezing | After freeze/thaw |
| (a) | 100 mg/mL | 1.1 | 1.0 |
| (b) | 150 mg/mL | 1.1 | 0.9 |

Both concentrations 100 mg/mL (a) and 150 mg/mL pembrolizumab (b) in 10 mM L-histidine/histidine HCl, 205 mM Trehalose dihydrate, 0.01% polysorbate 20, pH 5.5 are stable against freeze/thaw stress. Both products can be frozen without any increase of aggregation level which is a prerequisite for a long-term storage of e.g. drug substance.

### Example 3: Osmolality of Pembrolizumab in Concentrations of 100 mg/mL or 150 mg/mL in Histidine-Buffered Solution

Samples (a) and (b) produced in example 1 and shown in Table 16 were tested for osmolality. Object of this development is a pembrolizumab formulation which is suitable for both intravenous and subcutaneous application. Whereas formulations for the intravenous application route of pembrolizumab can comprise a wider range of osmolality, solutions injected subcutaneously should in best case be isotonic. Osmolality was determined by freezing point depression with an Osmomat 3000 from Gonotec, Germany as duplicate measurement.

TABLE 18
Osmolality of 100 mg/mL (a) and 150 mg/mL pembrolizumab
(b) in 10 mM L-histidine/histidine HCl, 205 mM Trehalose
dihydrate, 0.01% polysorbate 20, pH 5.5

| | | | Osmolality mean | RSD of |
|---|---|---|---|---|
| | | Osmolality | of triplicate | triplicates |
| Sample | | [mOsm/kg]] | [mPa*s] | [%] |
| 100 mg/mL | n1 | 374 | 374 | 0.0 |
| | n2 | 374 | | |
| 150 mg/mL | n1 | 463 | 464 | 0.2 |
| | n2 | 465 | | |

The osmolality data show that both the 100 mg/ml and the 150 mg/ml samples are suitable for subcutaneous administration.

### Example 4: Real Time and Accelerated Stability Study of Pembrolizumab in Concentrations of 100 mg/ml and 150 mL in Histidine Buffered Solution

A stability study was performed with samples (a) and (b) according to Table 16. Samples were stored at −70° C., 5° C. (target storage temperature), 25° C./60% relative humidity and 40° C./75% relative humidity for up to 3 months.

TABLE 19
Detailed information of stability program

| | T0 | 1 w | | | 1 month | | | 3 months | | | |
|---|---|---|---|---|---|---|---|---|---|---|---|
| Method | n.a. | 5° C. | 25° C. | 40° C. | 5° C. | 25° C. | 40° C. | −70° C. | 5° C. | 25° C. | 40° C. |
| cIEF | x | n.a. | n.a. | n.a. | x | n.a. | x | x | x | n.a. | x |
| cGE n.r. | x | n.a. | n.a. | n.a. | x | n.a. | x | x | x | n.a. | x |
| SE-HPLC | x | x | x | x | x | x | x | x | x | x | x |

Protein stability was determined by size exclusion chromatography (SE-HPLC) for the presence of high molecular weight species (HMWS), by non-reduced SDS-cGE for the presence of fragments (LMWS) and HMWS. Imaged capillary isoelectric focusing (icIEF) was used to detect modifications leading to charge heterogeneities.

Analysis of high molecular weight species (HMWS) by SE-HPLC The protein samples of the stability study were diluted with the corresponding formulation buffer to a concentration of 2.5 mg/mL and 20 μl of the dilution were applied onto a TSKgel G3000SWXL, (Tosoh, 300×7.8 mm, 5 μm) column to detect high molecular weight species of pembrolizumab.

The protein was eluted by isocratic elution using 0.1 M potassium phosphate buffer with 0.25 M potassium chloride (pH 5.6) at a flow rate of 0.5 mL/min at 25° C. Eluted species were detected at a wavelength of 280 nm and displayed on a graph showing the concentration of the eluted species vs. time. The elution profile showed a main peak with the non-aggregated protein and peaks of the protein representing higher molecular weight forms of the protein. The areas of all peaks were determined.

Table 20 shows the percentage of peak area for the HMWS in relation to the total peak area of the eluted species for the samples of the stability study shown in Table 19. Each sample was examined in duplicate measurements.

TABLE 20
Overview of HMWS determined via SE-HPLC

| | 100 mg/mL | 150 mg/mL |
|---|---|---|
| | pembrolizumab | pembrolizumab |
| | HMWS [%] | HMWS [%] |
| | Mean of two | Mean of two |
| Condition | measurements | measurements |
| T0 | 0.73% | 0.89% |
| 1 week 5° C. | 0.88% | 0.92% |
| 1 month 5° C. | 0.87% | 0.97% |
| 3 months 5° C. | 0.96% | 1.13% |
| 1 week 25° C. | 1.20% | 1.09% |
| 1 month 25° C. | 1.17% | 1.37% |
| 3 months 25° C. | 1.33% | 1.57% |
| 1 week 40° C. | 1.38% | 1.84% |
| 1 month 40° C. | 2.00% | 2.62% |
| 3 months 40° C. | 5.93% | 7.94% |
| 3 months -70° C. | 0.71% | 1.55% |

At target storage condition 5° C. generation of HMWS was very low, the products are stable against formation of aggregates. This is valid for both tested concentrations, for the formulation comprising 100 mg/mL pembrolizumab as well for the formulation comprising 150 mg/mL pembrolizumab. After a three-month storage HMWS just slightly increase compared to the starting point, in the 100 mg/mL sample from 0.73% to 0.96%, and in the 150 mg/mL sample by 0.89% to 1.13%. Beside real time storage also accelerated conditions led to very promising stability results, as HMWS were quantified in acceptable ranges for stress storage.

When storing the samples frozen at −70° C., the 100 mg/mL sample showed no increase of HMWS after 3 months, the 150 mg/mL sample showed just a slight increase to 1.55% HMWS.

### Detection of HMWS and LMWS by SDS-cGE Non-Reduced

Capillary gel electrophoresis was carried out based on the IgG Purity and Heterogeneity Analysis established by Beckman Coulter. The samples were diluted in SDS Sample Buffer pH 6.4-6.9 (10 mM citrate phosphate, 1% SDS) to a final concentration of 1 mg/mL for non-reduced analysis. Afterwards the thiol alkylating reagent N-Ethylmaleimide (NEM; 10 mM) was added to the sample mix to prevent fragmentation. Prior to analysis the sample was heat denatured at 70° C. for 10 min. For the cGE Method, separation was performed by forward injection in a neutral, bare fused silica capillary (20 cm effective length and 50 μm diameter) with a PA800 plus instrument from Beckman Coulter. After voltage forced application of the sample (5 kV for 20 s) into the capillary, protein separation was performed by applying a voltage of 15 kV for 30 min in case of reducing conditions and 15 kV for 40 min in case of non-reducing conditions. UV absorption was measured at 220 nm using the PDA detector and a 100×200 aperture. The capillary temperature was kept constant at 25° C. for all steps. The autosampler temperature was set to 15° C. Data were evaluated in terms of peak integration using the 32Karat software (Beckman Coulter). Peak areas were determined as velocity-corrected relative peak areas, considering the fact that in capillary electrophoresis early peaks migrate faster through the detector window than later peaks. Sample peak integration was performed in comparison to the electropherogram of a formulation buffer or pure water blank to identify and exclude non-protein-specific peaks.

Samples shown in Table 16 were analyzed as single measurement after incubation for 1 or 3 months at 5° C. or 40° C./75% relative humidity and frozen at −70° C.

According to Table 21 both tested formulations (with 100 mg/mL and 150 mg/mL pembrolizumab) were stable at storage for three months at target storage temperature 5° C., LMWS showed just very slight increases during this time period promising long term stability regarding the generation of LMWS tested by SDS-cGE non-reduced (100 mg/mL from 1.105% to 1.193%; 150 mg/mL from 1.116% to 1.170%). Negligible HMWS were detected in both formulations for this incubation period (100 mg/mL from 0.090% to 0.151%; 150 mg/mL from 0.139% to 0.166%). Also, when stored frozen at −70° C. no or negligible LMWS or HMWS were generated.

Storage at higher temperatures (1 month 40° C./75% RH) led to slightly increased values as expected for a storage at accelerated conditions, both tested formulations comprised LMWS and a slight increase of HMWS in a very comparable range.

TABLE 21
Overview of LMWS and HMWS determined via SDS-cGE non-reduced

| | 100 mg/mL pembrolizumab | | | 150 mg/mL pembrolizumab | | |
|---|---|---|---|---|---|---|
| | LMWS | Main Peak | HMWS | LMWS | Main Peak | HMWS |
| Condition | [%] | [%] | [%] | [%] | [%] | [%] |
| T0 | 1.105% | 98.804% | 0.090% | 1.116% | 98.744% | 0.139% |
| 1 month 5° C. | 1.148% | 98.713% | 0.139% | 1.142% | 98.659% | 0.198% |
| 3 months 5° C. | 1.193% | 98.656% | 0.151% | 1.170% | 98.664% | 0.166% |
| 1 month 40° C. | 3.412% | 96.143% | 0.445% | 3.392% | 95.818% | 0.790% |
| 3 months 40° C. | 9.190% | 89.966% | 0.845% | 9.256% | 89.585% | 1.158% |
| 3 months −70° C. | 1.148% | 98.763% | 0.088% | 1.086% | 98.837% | 0.077% |

Detection of Acidic Species and Basic Species by icIEF

The aim of imaged capillary isoelectric focusing (icIEF) is to determine the isoelectric point (pI) and the heterogeneity of charge isoforms of a protein that are caused by posttranslational modifications (PTM). The power of icIEF lies in the high resolving electropherograms allowing a precise and reproducible relative quantification of the charge isoforms.

Samples were rebuffered by ultrafiltration against ultrapure water and interfering buffer components were depleted from the samples using Detergent Removal Spin Columns (Pierce) according to the manufacturers' instructions. The protein concentration was determined by UV-measurement (Absorption at 280 nm). The rebuffered sample was diluted to 1 g/L with ultrapure water. 40 μg diluted sample (corresponds to a final protein concentration of 0.2 g/L) were mixed with 2% ampholytes (0.25% pH 9-11 and 3% 8-10.5), 0.35% methylcellulose, 4 M Urea and pI markers (8.18 and 9.99) as indicated. Focusing was performed in two steps (1 min at 1500 V and 10 min at 3000 V). Final analysis was carried out with the imaged CEsystem Maurice C (ProteinSimple). Data were evaluated by pI calibration of the electropherograms using the two internal pI markers and Compass for iCE software (ProteinSimple). Peak integration was performed using Empower software (Waters). Calculations with relative peak areas (e.g. for peak grouping) were performed using initial values for relative peak areas with three decimal places. The initial values used for calculations and the calculated results are reported rounded to one decimal place.

Samples were analyzed as single measurement during incubation for 3 months at 5° C. and 40° C./75% relative humidity.

According to Table 22 the 3-month data at 5° C. reveal excellent stability of pembrolizumab in both formulations (100 mg/mL and 150 mg/mL), as no significant changes acidic species and basic species were quantified. Also, when stored frozen at −70° C. no further acidic and basic species were generated. During storage at 40° C./75% relative humidity a shift to acidic species was detected as expected for a storage at accelerated conditions. This shift led to a decrease of main peak and basic species in both formulations.

TABLE 22
Overview of acidic species and basic species determined via cIEF

| | 100 mg/mL pembrolizumab | | | 150 mg/mL pembrolizumab | | |
|---|---|---|---|---|---|---|
| | Acidic | | Basic | Acidic | | Basic |
| Condition | species | Main Peak | species | species | Main Peak | species |
| T0 | 26.0% | 60.7% | 13.3% | 26.5% | 59.8% | 13.7% |
| 1 month 5° C. | 28.8% | 57.2% | 14.1% | 27.1% | 60.6% | 12.3% |
| 3 months 5° C. | 27.7% | 58.6% | 13.6% | 27.6% | 58.8% | 13.6% |
| 1 month 40° C. | 40.5% | 48.4% | 11.1% | 40.4% | 49.6% | 10.0% |
| 3 months 40° C. | 64.3% | 29.0% | 6.7% | 66.1% | 27.9% | 5.9% |
| 3 months −70° C. | 27.4% | 58.7% | 13.9% | 27.1% | 58.6% | 14.3% |

### Example 5: Viscosity Testing of High Concentration Samples in Histidine-Buffered Solution by Rheometry

The aim of this example was to measure the dynamic viscosity of pembrolizumab in formulations with different antibody concentrations. Both concentrations of pembrolizumab in 10 mM L-histidine/histidine HCl, 205 mM Trehalose dihydrate, 0.01% polysorbate 20, pH 5.5 as shown in Table 8 were analyzed, also a 25 mg/m sample in the identical formulation was analyzed, prepared by a 1:4 dilution of a 100 mg/mL sample with its formulation.

Dynamic viscosity of the three samples was determined on a Kinexus ultra plus rheometer at 20° C. and a shear rate of 250 s-1. For analyzing viscosity, methods and cone/plate geometry according to Table 23 were applied:

TABLE 23
Method setup for viscosity measurement

| | | | Sample | | Temperature/ | Measuring/ |
|---|---|---|---|---|---|---|
| Sample | | Geometry | volume | | shear rate | sampling time |
| 25 | mg/mL | CP1/40 | 310 | μL | 20° C./250 s⁻¹ | 3 min/2 sec |
| 100 | mg/mL | CP1/20 | 60 | μL | | |
| 150 | mg/mL | CP1/20 | 60 | μL | | |

All samples were analyzed as triplicate measurements and results are shown in Table 16. Formulations with 25 mg/mL and 100 mg/mL pembrolizumab showed very low dynamic viscosities of 1.463 mPa*s and 4.646 mPa*s respectively. The 150 mg/mL pembrolizumab formulation showed an increased but still acceptable viscosity of 17.88 mPa*s allowing ordinary DSP processes, filling of drug substance and drug product, and injectability by using a standard prefilled syringe for subcutaneous application.

TABLE 24
Dynamic viscosities obtained for three different
concentrations of Pembrolizumab

| | | | Viscosity of | Viscosity mean | RSD of |
|---|---|---|---|---|---|
| | | | single measurement | of triplicate | triplicates |
| Sample | | | [mPs*s] | [mPa*s] | [%] |
| 25 | mg/mL | n1 | 1.430 | 1.463 | 4.0 |
| | | n2 | 1.430 | | |
| | | n3 | 1.530 | | |
| 100 | mg/mL | n1 | 4.705 | 4.646 | 1.6 |
| | | n2 | 4.560 | | |
| | | n3 | 4.673 | | |
| 150 | mg/mL | n1 | 18.475 | 18.788 | 2.2 |
| | | n2 | 18.631 | | |
| | | n3 | 19.257 | | |

### Example 6: Quantification of Break Loose Force and Gliding Force when Using a Prefilled Syringe Suitable for Subcutaneous Injection

For testing the suitability of the highly concentrated formulations for subcutaneous administration, 1 mL of both formulations (containing 100 mg/mL and 150 mg/mL pembrolizumab) according to Table 16 were filled into a glass syringe (BD Neopak 1MILL 29G TW ½ 5B RNS BD260, Cat.-No. 47432910) with a nominal volume of 1 mL comprising a staked needle of 29 gauge. The syringe was vent tube stoppered (BD Hypak BSCF 1 MLL W4023 Flur DAIKYO LID, Cat.-No. 47284410).

After filling, the syringes were tested for injection forces simulating an injection with an injection speed of 190 mm/min by using a tensile testing machine (ZwickiLine 500 N, ZwickRoell GmbH & Co.KG, Ulm, Germany). The usage of a tensile machine enables the recording of an applied force in a force displacement plot. The injection force of a syringe can be divided into the break-loose force and the gliding force. Break-loose force is the force required for initiating the movement of the plunger, the gliding force (dynamic glide force) describes the force required to sustain the continues movement of the plunger.

A plunger rod was mounted to the stopper without moving the stopper, and after equilibrating the system to room temperature the measurement was performed.

TABLE 25
Injection forces of high concentrated pembrolizumab
filled in a prefilled syringe

| | Break-loose | Gliding force | Gliding force |
|---|---|---|---|
| Sample | force | (average) | (maximal force) |
| 100 mg/mL | 2.48N | 6.86N | 8.23N |
| 150 mg/mL | 1.74N | 10.59N | 14.17N |

The results (see Table 25) show a break-loose and gliding force (average and maximum) below 15 N for both formulations with 100 mg/mL and 150 mg/mL pembrolizumab when expelling the content. These values allow an easy and safe subcutaneous injection of the highly concentrated solutions into the patient.

### Example 7: Pembrolizumab in Concentrations of 100 mg/m and 150 mL in an Alternative Formulation Based on Citrate

Pembrolizumab will be transferred by UF/DF into a citrate buffered formulation shown in Table 26 and concentrated to target concentrations of 100 mg/mL and 150 mg/mL pembrolizumab.

TABLE 26
Detailed information of products used in example 7

| | Pembro- | Buffer | | | |
|---|---|---|---|---|---|
| Sample | lizumab | system | Stabilizer | Surfactant | pH |
| (a) | 100 mg/mL | 10 mM citric | 205 mM | 0.2 mg/mL | pH 5.5 |
| | | acid/sodium | trehalose | polysorbate | |
| | | citrate | dihydrate | 80 | |
| (b) | 150 mg/mL | 10 mM citric | 205 mM | 0.2 mg/mL | pH 5.5 |
| | | acid/sodium | trehalose | polysorbate | |
| | | citrate | dihydrate | 80 | |

After preparation samples will be treated by freeze/thaw cycles. In addition, a real time (2° C.-8° C.) and accelerated stability study (25° C., 40° C.) will be performed to analyze stability of the products against generation of HMWS by SE-HPLC. Viscosity testing of samples will be performed by using a rheometer, also the functionality by simulating a subcutaneous injection using a prefilled syringe will be tested by a tensile machine.

### Example 8: Sample Preparation of Highly Concentrated Pembrolizumab in a Citrate Buffered Solution

Pembrolizumab was transferred by UF/DF into a citrate buffered formulation shown in Table 27 and concentrated to target concentrations of 100 mg/mL and 150 mg/mL pembrolizumab.

TABLE 27
Detailed information of products used in example 8

| | Pembro- | Buffer | | | |
|---|---|---|---|---|---|
| Sample | lizumab | system | Stabilizer | Surfactant | PH |
| (c) | 100 mg/mL | 10 mM citric | 205 mM | 0.2 mg/mL | pH 5.5 |
| | | acid/sodium | trehalose | polysorbate | |
| | | citrate | dihydrate | 80 | |
| (d) | 150 mg/mL | 10 mM citric | 205 mM | 0.2 mg/mL | pH 5.5 |
| | | acid/sodium | trehalose | polysorbate | |
| | | citrate | dihydrate | 80 | |

Pembrolizumab was transferred by UF/DF and subsequent spiking of polysorbate 80 into a formulation containing 10 mM citric acid/sodium citrate, 205 mM Trehalose dihydrate, 0.2 mg/mL polysorbate 80, pH 5.5, and 100 mg/mL or 150 mg/mL pembrolizumab by the following steps:

15 grams of pembrolizumab at a concentration of 10.8 mg/mL were concentrated by UF/DF and transferred into a formulation containing 10 mM citric acid/sodium citrate, 205 mM Trehalose dihydrate, pH 5.5 to a target concentration of 120 mg/mL. For this step a Pellicon® 3 Cassette with Biomax® 30 kDa Membrane, D screen was used (Merck Millipore), providing a membrane load of approximately 15 g pembrolizumab/264 cm². As process condition 1 bar transmembrane pressure was applied with a 6 L/m²/min feed flow rate. Buffer exchange was carried out for ≤5 diafiltration volumes with formulation buffer without polysorbate 80.

After that the resulting solution containing 121.6 mg/mL pembrolizumab was sterile filtered by using a Sartopore 2 XLG 0.22 μm PES filter (filter area 210 cm²) without pre-flush of the filter. The final concentration of pembrolizumab was quantified to be 119.8 mg/mL with a very good step recovery of 93.5% compared to the starting material comprising 10.8 mg/mL pembrolizumab.

After that a 50:50 split of this solution was conducted. The first part was diluted with 10 mM L-citric acid/sodium citrate, 205 mM Trehalose dihydrate, pH 5.5 to a final concentration of 100 mg/mL pembrolizumab in 10 mM citric acid/sodium citrate, 205 mM Trehalose dihydrate, pH 5.5, and polysorbate 80 was added to provide a final concentration of 0.2 mg/mL polysorbate 80 in the solution.

The second part was transferred into a Vivaspin filter (Sartorius Stedim) using a 30 kDa PES membrane and the concentration was successfully increased to 161 mg/mL pembrolizumab. After that the concentration was adjusted to 150 mg/mL pembrolizumab by dilution with 10 mM citric acid/sodium citrate, 205 mM Trehalose dihydrate, pH 5.5, followed by sterile filtration using a bottle top vacuum filter (PES filter membrane area: 13.6 μm). Finally, polysorbate 80 was spiked into the formulation to a final concentration of 0.2 mg/mL.

Table 28 shows analytical results covering the different steps during preparation of the samples and the final processed samples comprising a pembrolizumab concentration of about 100 mg/mL or 150 mg/mL. The process was very gentle and smooth, HMWS determined by SE-HPLC showed just a slight increase from 0.85% HMWS to 1.20% in the 100 mg/mL pembrolizumab sample and to 1.31% in the 150 mg/mL sample. Other impurities like LMWS (quantified by CE-SDS non-reduced) and acidic and basic species (quantified by HP-CEX) were also in an acceptable range and not altered by the processing steps. With getting the solutions successfully sterile filtered through a 0.22 μm filter with negligible loss of material, pembrolizumab in the described citrate buffered solution is a very good formulation candidate suitable for achieving high protein concentrations in a scalable manufacturing process. All methods are described in Example 4, 5 and 6.

TABLE 28
Preparation of highly concentrated pembrolizumab in 10 mM citric acid/sodium citrate, 205 mM Trehalose
dihydrate, 0.2 mg/mL polysorbate 80, pH 5.5 by UF/DF and characterization of samples

| | | Concentration | | | Δ HMWS increase | |
|---|---|---|---|---|---|---|
| | | pembrolizumab | | HMWS | compared to Sample | |
| Sample ID | Description | (UV280) | pH (22° C.) | (SE-HPLC) | before UF/DF | Main Peak (SE-HPLC) |
| SKL001.01- | Sample before UF/DF | 10.79 mg/mL | n.a. | 0.85% | n.a. | 99.15% |
| UFDF-S | | | | | | |
| SKL001.01- | Process sample 20 mg/mL | 19.97 mg/mL | 5.46 | 0.93% | 0.08% | 99.07 |
| DF-E | | | | | | |
| SKL001.01- | Process sample 40 mg/mL | 38.48 mg/mL | n.a. | 0.98% | 0.13% | 99.02% |
| UF2-E1 | | | | | | |
| SKL001.01- | Process sample 80 mg/mL | 73.48 mg/mL | n.a. | 1.03% | 0.18% | 98.97% |
| UF2-E2 | | | | | | |
| SKL001.01- | Sample after UF/DF | 121.55 mg/mL | 5.47 | 1.13% | 0.28% | 98.87% |
| UF2-E | | | | | | |
| SKL001.01- | Sample after Vivaspin concentration | 160.95. mg/mL | 5.49 | 1.33% | 0.48% | 98.67% |
| UF3-E | | | | | | |
| SKL001.01- | Sample at target 100 mg/mL, | 101.26 mg/mL | 5.47 | 1.20% | 0.35% | 98.9% |
| DS-E | formulated in 10 mM citric acid/sodium | | | | | |
| | citrate, 205 mM Trehalose dihydrate, | | | | | |
| | 0.2 mg/mL polysorbate 80, pH 5.5, 0.22 | | | | | |
| | μm filtrated, sample (c) | | | | | |
| SKL001.02- | Sample at target 150 mg/mL, | 149.76 mg/mL | 5.49 | 1.31% | 0.46% | 98.9% |
| DS-E | formulated in 10 mM citric acid/sodium | | | | | |
| | citrate, 205 mM Trehalose dihydrate, | | | | | |
| | 0.2 mg/mL polysorbate 20, pH 5.5, 0.22 | | | | | |
| | μm filtrated, sample (d) | | | | | |

### Example 9: Osmolality of Pembrolizumab in Concentrations of 100 mg/mL or 150 mg/mL in Citrate-Buffered Solution

The osmolality of the samples (c) and (d) produced in example 8 and shown in Table 27 was determined. Object of this development is a pembrolizumab formulation which is suitable for both intravenous and subcutaneous application. Whereas formulations for the intravenous application route of pembrolizumab can comprise a wider range of osmolality, solutions to be injected subcutaneously should in best case be isotonic. Osmolality was determined by freezing point depression with an Osmomat 3000 from Gonotec, Germany as duplicate measurement.

TABLE 29
Osmolality of 100 mg/mL (c) and 150 mg/mL pembrolizumab
(d) in 10 mM citric acid/sodium citrate, 205 mM Trehalose
dihydrate, 0.2 mg/mL polysorbate 80, pH 5.5

| | | | Osmolality mean | RSD of |
|---|---|---|---|---|
| | | Osmolality | of duplicate | triplicates |
| Sample | | [mOsm/kg]] | [mPa*s] | [%] |
| 100 mg/mL | n1 | 298 | 298 | 0.00 |
| | n2 | 298 | | |
| 150 mg/mL | n1 | 362 | 363 | 0.14 |
| | n2 | 363 | | |

The osmolality data shown in Table 29 demonstrate that both the 100 mg/ml and the 150 mg/ml samples, all formulated in 10 mM citric acid/sodium citrate, 205 mM Trehalose dihydrate, 0.2 mg/mL polysorbate 80, pH 5.5, are very suitable for subcutaneous administration.

### Example 10: Real Time and Accelerated Stability Study of Pembrolizumab in Concentrations of 100 mg/ml and 150 mL in Citrate Buffered Solution

A stability study was performed with samples (c) and (d), all formulated in 10 mM citric acid/sodium citrate, 205 mM Trehalose dihydrate, 0.2 mg/mL polysorbate 80, pH 5.5, according to Table 30. Samples were stored at −70° C., 5° C. (target storage temperature), 25° C./60% relative humidity and 40° C./75% relative humidity for up to 3 months.

TABLE 30
Detailed information of stability program

| | T0 | 1 w | | | 1 month | | | 3 months | | | |
|---|---|---|---|---|---|---|---|---|---|---|---|
| Method | n.a. | 5° C. | 25° C. | 40° C. | 5° C. | 25° C. | 40° C. | −70° C. | 5° C. | 25° C. | 40° C. |
| cIEF | x | n.a. | n.a. | n.a. | x | n.a. | x | x | x | n.a. | x |
| cGE n.r. | x | n.a. | n.a. | n.a. | x | n.a. | x | x | x | n.a. | x |
| SE-HPLC | x | x | x | x | x | x | x | x | x | x | x |

Protein stability was determined by size exclusion chromatography (SE-HPLC) for the presence of high molecular weight species (HMWS), by non-reduced SDS-cGE for the presence of fragments (LMWS) and HMWS. Imaged capillary isoelectric focusing (icIEF) was used to detect modifications leading to charge heterogeneities.

All methods were performed according to example 4.

### Analysis of High Molecular Weight Species (HMWS) by SE-HPLC

Table 31 shows the percentage of peak area for the HMWS and main peak in relation to the total peak area of the eluted species for the samples of the stability study shown in Table 30. Each sample was examined in duplicate measurements.

TABLE 31
Overview of HMWS and LMWS determined via SE-HPLC

| | 100 mg/mL | | 150 mg/mL | |
|---|---|---|---|---|
| | pembrolizumab | | pembrolizumab | |
| | Sample (c) | | Sample (d) | |
| | | Main | | Main |
| Condition | HMWS [%] | peak [%] | HMWS [%] | peak [%] |
| T0 | 0.79 | 99.21 | 0.80 | 99.20 |
| 1 week 5° C. | 0.88 | 99.12 | 0.88 | 99.12 |
| 1 month 5° C. | 1.04 | 98.96 | 1.07 | 98.93 |
| 3 months 5° C. | 1.07 | 98.93 | 1.33 | 98.67 |
| 1 week 25° C. | 1.05 | 98.95 | 1.25 | 98.75 |
| 1 month 25° C. | 1.30 | 98.70 | 1.49 | 98.51 |
| 3 months 25° C. | 1.65 | 98.35 | 2.07 | 97.93 |
| 1 week 40° C. | 1.41 | 98.59 | 1.74 | 98.26 |
| 1 month 40° C. | 2.57 | 97.43 | 2.93 | 96.80 |
| 3 months 40° C. | 4.14 | 95.58 | 5.49 | 94.17 |
| 3 months -70° C. | 0.75 | 99.25 | 0.84 | 99.16 |

At target storage condition 5° C. generation of HMWS was very low, the products are stable against formation of aggregates. This is valid for both tested concentrations, for the formulation comprising 100 mg/mL pembrolizumab as well as for the formulation comprising 150 mg/mL pembrolizumab. After a three-month storage HMWS just slightly increase compared to the starting point, in the 100 mg/mL sample from 0.79% to 1.07%, and in the 150 mg/mL sample by 0.80% to 1.33%. Besides real time storage also accelerated conditions led to very promising stability results, as HMWS were quantified in acceptable ranges for stress storage.

When storing the samples frozen at −70° C., 100 mg/mL sample as well 150 mg/mL sample showed no increase of HMWS after 3 months, the results are within assay variation. Both products can be frozen without any increase of aggregation level which is a prerequisite for a long-term storage of e.g. drug substance.

### Detection of HMWS and LMWS by SDS-cGE Non-Reduced

Samples (c) and (d) shown in Table 27 were analyzed as single measurement after incubation for 1 or 3 months at 5° C. or 40° C./75% relative humidity, or frozen at −70° C. for 3 months.

According to Table 32 both tested formulations (with 100 mg/mL and 150 mg/mL pembrolizumab) were stable at storage for three months at target storage temperature 5° C. LMWS showed no or just a very slight increase during this time period promising long term stability regarding the generation of LMWS tested by SDS-cGE non-reduced (100 mg/mL: no increase; 150 mg/mL from 1.004% to 1.144%). Slightly increasing amounts of HMWS were detected in both formulations for this incubation period (100 mg/mL from 0.068% to 0.312%; 150 mg/mL from 0.106% to 0.450%). Also, when stored frozen at −70° C. no or negligible LMWS or HMWS were generated.

Storage at higher temperatures (1 month and 3 months at 40° C./75% RH) led to increased values as expected for a storage at accelerated conditions. Both tested formulations comprised LMWS and a slight increase of HMWS in a very comparable range.

TABLE 32
Overview of LMWS and HMWS determined via SDS-cGE non-reduced

| | 100 mg/mL pembrolizumab | | | 150 mg/mL pembrolizumab | | |
|---|---|---|---|---|---|---|
| | LMWS | Main Peak | HMWS | LMWS | Main Peak | HMWS |
| Condition | [%] | [%] | [%] | [%] | [%] | [%] |
| T0 | 1.080 | 98.851 | 0.068 | 1.004 | 98.891 | 0.106 |
| 1 month 5° C. | 1.099 | 98.720 | 0.181 | 1.141 | 98.685 | 0.173 |
| 3 months 5° C. | 1.035 | 98.653 | 0.312 | 1.144 | 98.405 | 0.450 |
| 1 month 40° C. | 2.865 | 96.077 | 1.059 | 3.042 | 95.499 | 1.458 |
| 3 months 40° C. | 7.471 | 90.158 | 2.370 | 7.243 | 89.972 | 2.788 |
| 3 months −70° C. | 1.057 | 98.788 | 0.157 | 1.144 | 98.679 | 0.178 |

Detection of Acidic Species and Basic Species by icIEF

Samples according to Table 27 were analyzed as single measurement after incubation for 1 or 3 months at 5° C. or 40° C./75% relative humidity, or frozen at −70° C. for 3 months.

According to Table 33 the 3-month data at 5° C. reveal excellent stability of pembrolizumab in both formulations (100 mg/mL and 150 mg/mL), as no significant changes in acidic species and basic species were quantified. Also, when stored frozen at −70° C. no additional acidic and basic species were generated. During storage at 40° C./75% relative humidity a shift to acidic species was detected as expected for a storage at accelerated conditions. This shift led to a decrease of main peak and basic species in both formulations.

TABLE 33
Overview of acidic species and basic species determined via cIEF

| | 100 mg/mL pembrolizumab | | | 150 mg/mL pembrolizumab | | |
|---|---|---|---|---|---|---|
| | sample (c) | | | sample (d) | | |
| | Acidic | Main | Basic | Acidic | Main | Basic |
| Condition | species [%] | Peak [%] | species [%] | species [%] | Peak [%] | species [%] |
| T0 | 26.739 | 55.070 | 18.191 | 26.033 | 55.394 | 18.573 |
| 1 month 5° C. | 24.084 | 56.938 | 18.978 | 29.121 | 53.292 | 17.586 |
| 3 months 5° C. | 24.597 | 57.396 | 18.007 | 24.985 | 56.249 | 18.767 |
| 1 month 40° C. | 40.701 | 45.762 | 13.538 | 38.816 | 47.871 | 13.311 |
| 3 months 40° C. | 61.065 | 28.478 | 10.458 | 60.207 | 28.223 | 11.570 |
| 3 months −70° C. | 24.676 | 56.319 | 19.004 | 24.674 | 56.308 | 19.019 |

### Example 5: Viscosity Testing of High Concentration Samples in Citrate-Buffered Solution by Rheometry

The aim of this example was to measure the dynamic viscosity of pembrolizumab in formulations with different antibody concentrations. Both concentrations of pembrolizumab in 10 mM citric acid/sodium citrate, 205 mM Trehalose dihydrate, 0.2 mg/mL polysorbate 80, pH 5.5 as shown in Table 27 were analyzed.

Dynamic viscosity of the two samples was determined on a Kinexus ultra plus rheometer at 20° C. and a shear rate of 250 s-1. For analyzing viscosity, methods and cone/plate geometry according to Table 34 were applied:

TABLE 34
Method setup for viscosity measurement

| | | Sample | Temperature/ | Measuring/ |
|---|---|---|---|---|
| Sample | Geometry | volume | shear rate | sampling time |
| 100 mg/mL | CP1/20 | 60 μL | 20° C./250 s⁻¹ | 3 min/2 sec |
| 150 mg/mL | CP1/20 | 60 μL | 20° C./250 s⁻¹ | 3 min/2 sec |

All samples according to Table 27 formulated in 10 mM citric acid/sodium citrate, 205 mM Trehalose dihydrate, 0.2 mg/mL polysorbate 80, pH 5.5 were analyzed as triplicate measurements and results are shown in Table 35. The formulation with 100 mg/mL pembrolizumab showed a very low dynamic viscosity of 5.045 mPa*s. The 150 mg/mL pembrolizumab formulation showed an increased but still acceptable viscosity of 19.587 mPa*s allowing ordinary DSP processes, filling of drug substance and drug product, and injectability by using a standard prefilled syringe for subcutaneous application.

TABLE 35
Dynamic viscosities obtained for two different
concentrations of Pembrolizumab

| | | Viscosity of | Viscosity mean | RSD of |
|---|---|---|---|---|
| | | single measurement | of triplicate | triplicates |
| Sample | | [mPs*s] | [mPa*s] | [%] |
| 100 mg/mL | n1 | 4.957 | 5.045 | 2.5 |
| sample (c) | n2 | 5.190 | | |
| | n3 | 4.988 | | |
| 150 mg/mL | n1 | 19.665 | 19.587 | 2.0 |
| sample (d) | n2 | 19.161 | | |
| | n3 | 19.936 | | |

### Example 6: Quantification of Break Loose Force and Gliding Force by Simulating a Subcutaneous Injection of 100 mg/mL and 150 mg/mL Pembrolizumab in 10 mM Citric Acid/Sodium Citrate, 205 mM Trehalose Dihydrate, 0.2 mg/mL Polysorbate 80, pH 5.5

For testing the suitability of the highly concentrated formulations for subcutaneous administration, 1 mL of both formulations (containing 100 mg/mL and 150 mg/mL pembrolizumab in 10 mM citric acid/sodium citrate, 205 mM Trehalose dihydrate, 0.2 mg/mL polysorbate 80, pH 5.5) according to Table 27 were filled into a glass syringe (BD Neopak 1MILL 29G TW ½ 5B RNS BD260, Cat.-No. 47432910) with a nominal volume of 1 mL comprising a staked needle of 29 gauge. The syringe was vent tube stoppered (BD Hypak BSCF 1MLL W4023 Flur DAIKYO LID, Cat.-No. 47284410).

After filling, the syringes were tested for injection forces simulating an injection with an injection speed of 190 mm/min by using a tensile testing machine (ZwickiLine 500 N, ZwickRoell GmbH & Co.KG, Ulm, Germany). The usage of a tensile machine enables the recording of an applied force in a force displacement plot. The injection force of a syringe can be divided into the break-loose force and the gliding force. Break-loose force is the force required for initiating the movement of the plunger, the gliding force (dynamic glide force) describes the force required to sustain the continues movement of the plunger.

A plunger rod was mounted to the stopper without moving the stopper, and after equilibrating the system to room temperature the measurement was performed.

TABLE 36
Injection forces of highly concentrated pembrolizumab
filled in a prefilled syringe

| | | Break-loose | Break-loose | Gliding | Gliding |
|---|---|---|---|---|---|
| | | force | force | Force max | Force max |
| Sample | | [N] | (mean) [N] | [N] | (mean) [N] |
| 100 mg/mL | n1 | 3.06 | 3.0 | 8.08 | 7.9 |
| sample (c) | n2 | 2.89 | | 7.82 | |
| 150 mg/mL | n1 | 2.71 | 2.7 | 14.67 | 14.6 |
| sample (d) | n2 | 2.74 | | 14.60 | |

The results (see Table 36) show a break-loose and gliding force (average and maximum) below 15 N for both formulations with 100 mg/mL and 150 mg/mL pembrolizumab when expelling the syringe content. These values allow an easy and safe subcutaneous injection of the highly concentrated solutions into the patient.

1. A liquid pharmaceutical composition comprising:
(a) an anti-human PD1 antibody;
(b) one or more amino acids other than histidine;
(c) a non-ionic surfactant; and
(d) citrate buffer, wherein the pH of the composition is between 5.0 and 5.8.

2. The pharmaceutical composition of claim 1, wherein the citrate buffer is present in a concentration of 1 mM to 50 mM.

3. The pharmaceutical composition of claim 1, wherein the non-ionic surfactant is polysorbate 20 or polysorbate 80.

4. The pharmaceutical composition of claim 1, wherein the non-ionic surfactant is present in a concentration of 0.1 mg/ml to 0.4 mg/ml.

5. The pharmaceutical composition of claim 1, wherein the amino acid is arginine and/or lysine.

6. The pharmaceutical composition of claim 1, comprising arginine in a concentration of 100 mM to 350 mM or 250 mM to 350 mM.

7. The pharmaceutical composition of claim 1, comprising lysine in a concentration of 100 mM to 300 mM.

8. The pharmaceutical composition of claim 1, comprising arginine and lysine, wherein the total concentration of arginine and lysine is 100 mM to 350 mM.

9. The pharmaceutical composition of claim 1, wherein the anti-human PD1 antibody is pembrolizumab.

10. The pharmaceutical composition of claim 1, wherein the anti-human PD1 antibody is present in a concentration of 25 to 80 mg/ml.

11. A liquid pharmaceutical composition consisting of citrate buffer, arginine and/or lysine, polysorbate 20 or polysorbate 80, pembrolizumab and water for injection and having a pH of 5.0 to 5.8.

12. The liquid pharmaceutical composition of claim 11, consisting of:
(a) 5 or 10 mM citrate buffer, 300 mM arginine, 0.2 mg/ml polysorbate 80, 25 mg/ml pembrolizumab and water for injection and having a pH of 5.0 to 5.8; or
(b) 10 mM citrate buffer, 250 mM lysine, 0.2 mg/ml polysorbate 80, 25 mg/ml pembrolizumab and water for injection and having a pH of 5.0 to 5.8; or
(c) 10 mM citrate buffer, 150 mM arginine, 150 mM lysine, 0.2 mg/ml polysorbate 80, 25 mg/ml pembrolizumab and water for injection and having a pH of 5.0 to 5.8.

13.-17. (canceled)

18. A method for treating a subject having cancer, comprising administering to the subject the pharmaceutical composition of claim 1.

19. The pharmaceutical composition of claim 1, wherein the pH of the composition is 5.5.

20. The pharmaceutical composition of claim 1, wherein the citrate buffer is present in a concentration of 5 mM or 10 mM.

21. The pharmaceutical composition of claim 1, wherein the non-ionic surfactant is polysorbate 80.

22. The pharmaceutical composition of claim 1, wherein the non-ionic surfactant is present in a concentration of 0.2 mg/ml.

23. The pharmaceutical composition of claim 1, wherein the anti-human PD1 antibody is present in a concentration of 25 mg/ml.

24. The method of claim 18, wherein the cancer is melanoma, non-small cell lung carcinoma, classical Hodgkin lymphoma, urothelial carcinoma, head and neck squamous cell carcinoma, renal cell carcinoma, colorectal cancer, small cell lung cancer, microsatellite instability-high or mismatch repair deficient cancer, primary mediastinal large B-cell lymphoma, gastric or gastroesophageal junction adenocarcinoma, hepatocellular carcinoma, Merkel cell carcinoma, endometrial carcinoma, tumor mutational burden-high cancer, cutaneous squamous cell carcinoma, triple-negative breast cancer or cervical cancer.

25. A method for treating a subject having cancer, comprising administering to the subject the pharmaceutical composition of claim 11.
